(19) United States
(12) Patent Application Publication
(10) Pub. No.: US 2013/0217585 A1
(43) Pub. Date: Aug. 22, 2013

(54) Quantitative Total Definition of Biologically Active Sequence Elements

(71) Applicants: Lawrence A. Chasin, Leonia, NJ (US); Shengdong Ke, New York, NY (US)

(72) Inventors: Lawrence A. Chasin, Leonia, NJ (US); Shengdong Ke, New York, NY (US)

(73) Assignee: THE TRUSTEES OF THE UNIVERSITY OF COLUMBIA IN THE CITY OF NEW YORK, New York, NY (US)

(21) Appl. No.: 13/818,777

(22) PCT Filed: Aug. 25, 2011
(86) PCT No.: PCT/US2011/049098

§ 371 Date: Feb. 25, 2013

Related U.S. Application Data
(60) Provisional application No. 61/376,805, filed on Aug. 25, 2010.

(51) Int. Cl. C12Q 1/68

(52) U.S. Cl. CPC C12Q 1/6874
USPC 506/2

(57) ABSTRACT

A method and apparatus include preparing a library of molecules that can be sequenced. The library includes multiple instances of each possible member of a k-mer. The library is sequenced to determine the relative frequency of each member of the k-mer in the library. The library is contacted with a biochemical system. A population of output molecules is sequenced to determine the relative frequency of each member of the k-mer in the population of output molecules. Each output molecule is related to a product of a process of the biochemical system and carries a k-mer related to a corresponding k-mer of a library molecule involved in the process. Effectiveness of each member of the k-mer is determined based on the relative frequency of each member of the k-mer in the population of output molecules and the relative frequency of the corresponding k-mer in the library.

### CROSS-REFERENCE TO RELATED APPLICATIONS

This application claims benefit of Provisional Appln. 61/376,805, filed Aug. 25, 2010, the entire contents of which are hereby incorporated by reference as if fully set forth herein, under 35 U.S.C. §119(e).

### STATEMENT OF GOVERNMENTAL INTEREST

This invention was made with Government support under Contract No. NIH RO1 GM072740 awarded by the National Institutes of Health. The Government has certain rights in the invention.

### BACKGROUND OF THE INVENTION

Discovering the significance of particular sequences among various nucleic acids in biological systems is an object of ongoing research to understand and control such systems, including viruses, bacteria, cells, tissues and entire organisms.

Massively Parallel Sequencing (MPS) approaches such as those now in wide commercial use (Illumina/Solexa, Roche/454 Pyrosequencing, and ABI SOLiD) are attractive tools for sequencing. Typically, MPS methods can only obtain short read lengths (100 base pairs, bp, with IIlumina platforms to a maximum of 200-300 bp by 454 Pyrosequencing). Sanger methods, on the other hand, achieve longer read lengths of approximately 800 bp (typically 500-600 bp with non-enriched DNA). MPS has been used to identify successful binding sites for certain splicing factors. (See for example, Sanford, J. R. et al. Splicing factor SFRS 1 recognizes a functionally diverse landscape of RNA transcripts. Genome Res, v. 19, 381-94, 2009, the entire contents of this and all subsequent references cited herein or in the Appendix are hereby incorporated by reference as if fully set forth herein, except in so far as terms are used therein in conflict with the definition of such terms herein).

In other approaches, systematic evolution of ligands by exponential enrichment (SELEX) has been used to determine successful splicing factors in messenger ribonucleic acid (mRNA). (See, for example, Smith, P. J. et al. An increased specificity score matrix for the prediction of SF2/ASF-specific exonic splicing enhancers. Hum Mol Genet v.15, 2490-508,2006); and Reid, D. C. et al. Next-generation SELEX identifies sequence and structural determinants of splicing factor binding in human pre-mRNA sequence. RNA v.15, 2385-2397, 2009.)

### SUMMARY OF THE INVENTION

With the advent of affordable high throughput sequencing, it has become possible to carry out in vivo functional selections without iterations and on a scale that allows exhaustive testing of all possible k-mer sequences for k in the range of k=5 to k=8. It is anticipated that further advancements will allow exhaustive testing of all possible k-mer sequences for even larger values of k, such as k=10. Techniques are provided for taking advantage of such exhaustive testing for quantitative total definition of biologically active sequence elements.

According to one set of embodiments, a method comprises preparing a library of molecules that can be sequenced. The library includes multiple instances of each possible member of a k-mer. The method further comprises sequencing the library to determine the relative frequency of each member of the k-mer in the library. The method further comprises contacting the library with a biochemical system. The method yet further includes sequencing a population of output molecules to determine the relative frequency of each member of the k-mer in a population of output molecules. Each output molecule is related to a product of a process of the biochemical system and carries a k-mer related to a corresponding k-mer of a library molecule involved in the process. The method also includes determining effectiveness of each member of the k-mer based on the relative frequency of each member of the k-mer in the population of output molecules and the relative frequency of the corresponding k-mer in the library. According to some embodiments, the method includes identifying a particular set of one or more members of the k-mer in the library based on an effectiveness determined for the particular set.

According to another set of embodiments, a computer-readable storage medium carries one or more sequences of instructions, wherein execution of the one or more sequences of instructions by one or more processors causes an apparatus to determine a relative frequency of each member of a k-mer in a population of library molecules. The apparatus is further caused to determine the relative frequency of each member of the k-mer in a population of output molecules. Each output molecule is related to a product of a process of a biochemical system and carries a k-mer related to a corresponding k-mer of a library molecule involved in the process. The apparatus is further caused to determine effectiveness of each member of the k-mer based on the relative frequency of each member of the k-mer in the population of output molecules and the relative frequency of the corresponding k-mer in the library.

According to another set of embodiments, an apparatus comprises means for determining a relative frequency of each member of a k-mer in a population of library molecules. The apparatus further comprises means for determining the relative frequency of each member of the k-mer in a population of output molecules. Each output molecule is related to a product of a process of a biochemical system and carries a k-mer related to a corresponding k-mer of a library molecule involved in the process. The apparatus further comprises means for determining effectiveness of each member of the k-mer based on the relative frequency of each member of the k-mer in the population of output molecules and the relative frequency of the corresponding k-mer in the library.

According to various other sets of embodiments, a molecule or mixture of molecules is identified according to the above method, wherein the molecule is a nucleic acid or peptide or protein. Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a diagram that illustrates an example process for quantitative total definition of biologically active sequence elements, according to an embodiment;

FIG. 2 is a flow diagram that illustrates an example method for quantitative total definition of biologically active sequence elements, according to an embodiment;

FIG. 3A is a diagram that illustrates a DNA molecule of a population of library molecules used as input to a gene splicing process, according to an embodiment;

FIG. 3B is a diagram that illustrates example synthesis of the DNA molecule of a population of library molecules in relation to an example output molecule that results from a splicing process, according to an embodiment;

FIG. 3C is a diagram that illustrates an example process for quantitative total definition of gene splicing active sequence elements, according to an embodiment;

FIG. 4A is a graph that illustrates an example relative frequency of occurrence of 4096 members of a 6-mer in a population of input library molecules and in a population of spliced messenger RNA product molecules, according to an embodiment;

FIG. 4B is a graph that illustrates an example relative frequency of occurrence of 65,536 members of a 8-mer in a population of input library molecules and in a population of spliced messenger RNA product molecules, according to an embodiment;

FIG. 5A is a graph that illustrates an example relative frequency of occurrence of 4096 members of a 6-mer in two populations of input library molecules, according to an embodiment;

FIG. 5B is a graph that illustrates an example relative frequency of occurrence of 4096 members of a 6-mer in two populations of output molecules, according to an embodiment;

FIG. 5C is a graph that illustrates an example relative frequency of occurrence of 65,536 members of a 8-mer in two populations of input library molecules, according to an embodiment;

FIG. 5D is a graph that illustrates an example relative frequency of occurrence of 65,536 members of a 8-mer in two populations of output molecules, according to an embodiment;

FIG. 6 is a graph that illustrates an example distribution of gene splicing enrichment index (EI) among 4096 members of a 6-mer, where an EI is a ratio of relative frequency of a member of a 6-mer in a population of output molecules to the relative frequency of the same member of the 6-mer in the population of library molecules, according to an embodiment;

FIG. 7 is a graph that illustrates a relationship between a rate of inclusion of an exon in a spliced mRNA molecule based on enrichment index EI compared to an observed rate of inclusion, according to an embodiment;

FIG. 8 is a block diagram that illustrates a computer system upon which an embodiment of the invention may be implemented;

FIG. 9 is a block diagram that illustrates a chip set upon which an embodiment of the invention may be implemented

FIG. 10A and FIG. 10B are block diagrams that illustrate example different locations for each k-mer, according to an embodiment;

FIG. 11A is a graph that illustrates similar effectiveness of k-mers in two different locations, according to an embodiment;

FIG. 11B is a graph that illustrates dissimilar effectiveness of k-mers in two different locations, according to an embodiment;

FIG. 12A is a diagram that illustrates example overlapping k-mers changed by substitution of one k-mer in one location, according to an embodiment;

FIG. 12B is a diagram that illustrates example multiple occurrences of one k-mer in different locations, according to an embodiment;

FIG. 13 is a flow diagram that illustrates an example method for determining context adjusted effectiveness of biologically active sequence elements, according to an embodiment;

FIG. 14A is a graph that illustrates example average effectiveness scores of enhancing sequences, silencing sequences and neutral sequences, according to a splicing embodiment; and

FIG. 14B is a graph that illustrates example relationship between LEIsc values and predicted effectiveness, according to a splicing embodiment.

### DETAILED DESCRIPTION

A method and apparatus are described for quantitative total definition of biologically active nucleotide or amino acid sequence elements. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Deoxyribonucleic acid (DNA) is a self replicating, usually double-stranded long molecule that encodes other shorter molecules, such as proteins, used to build and control all living organisms. DNA is composed of repeating chemical units known as “nucleotides” or “bases.” There are four bases: adenine, thymine, cytosine, and guanine, represented by the letters A, T, C and G, respectively. Adenine on one strand of DNA always binds to thymine on the other strand of DNA; and guanine on one strand always binds to cytosine on the other strand and such bonds are called base pairs. Any order of A, T, C and G is allowed on one strand, and that order determines the complementary order on the other strand. The actual order determines the function of that portion of the DNA molecule. Information on a portion of one strand of DNA can be captured by ribonucleic acid (RNA) that also comprises a chain of nucleotides in which uracil (U) replaces thymine (T). Determining the order, or sequence, of bases on one strand of DNA or RNA is called sequencing. A portion of length k bases of a strand is called a k-mer; and specific short k-mers are called oligonucleotides or oligomers or “oligos” for short.

Some example embodiments of the invention are described below in the context of identifying the effect of nucleotide members of a 6-mer in a gene on the splicing of exons into mRNA. However, the invention is not limited to this context. In other embodiments the effect or function of a k-mer in DNA and RNA molecules or in peptides and proteins is determined for the same or other biochemical processes, including biological processes, for k in the range from about 5 to about 8 or more. In various embodiments, such biochemical processes include gene activation, mRNA processing or transport, mRNA degradation, protein binding, and enzymatic activity, among others, alone or in some combination.

### 1. Definitions

The terms used herein have the meanings in the following table.

| | |
|---|---|
| k-mer | a sequence of k nucleotides or amino acids at a particular location on a |
| | type of molecule |
| k-mer member | A molecule having a unique sequence within the k-mer |
| library | a population of molecules that can be sequenced and that has a |
| | particular distribution of k-mer members including at least one |
| | occurrence of each member of the k-mer. Library is used |
| | interchangeably with “input library” and “population of library |
| | molecules.” |
| biochemical process | a process involving one or more biologically active molecules |
| | including biological processes |
| biochemical system | a system of constituents involved in one or more biochemical |
| | processes |
| product molecule | a molecule that is produced by a process of the biochemical system |
| | and has a portion related to the k-mer in the library |
| derivative molecule | a molecule that is derived from a product molecule and includes a |
| | k-mer related to the k-mer in the library; for example, the product of |
| | an enzymatic reaction. |
| output molecule | a product molecule or derivative molecule that is sequenced to find a |
| | member of a k-mer related to a corresponding k-mer in the library |
| substantively | two or more populations of molecules that exhibit identical |
| identical | distributions of members of a k-mer with R² greater than about |
| populations | 0.3, where R² is the coefficient of determination (or proportion of |
| | explained variance) |

### 2. Overview

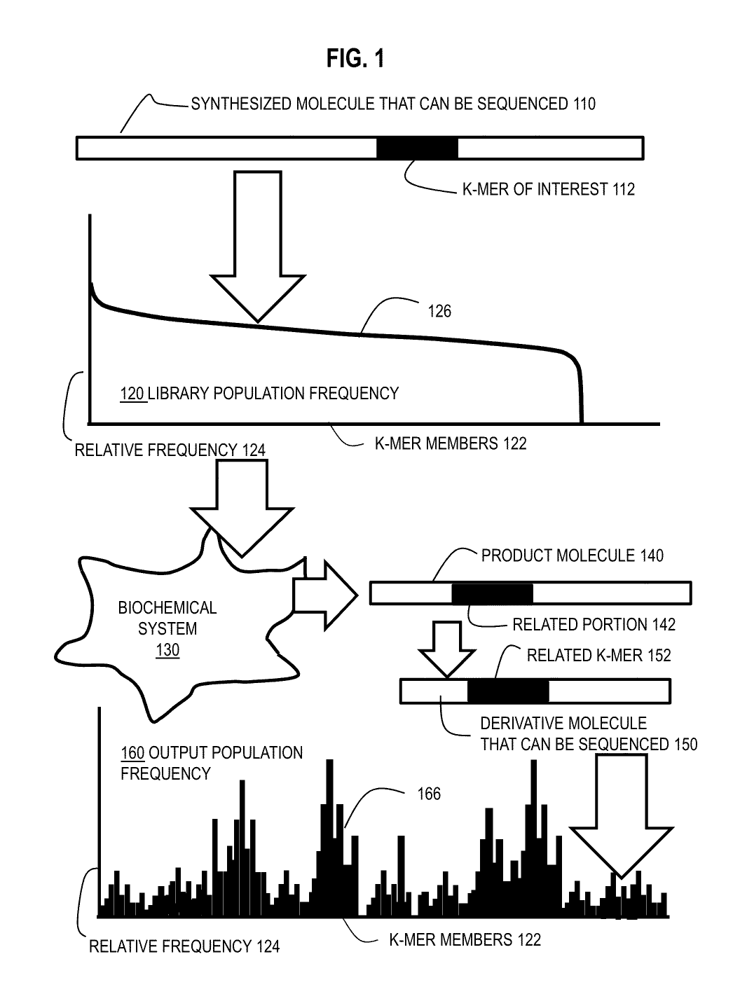

FIG. 1 is a diagram that illustrates an example process for quantitative total definition of biologically active sequence elements, according to an embodiment. A synthesized molecule 110 that can be sequenced (e.g., for which a nucleotide sequence or amino acid sequence can be determined) includes a k-mer of interest 112 at a particular location. In various embodiments, the synthesized molecule 110 is a single-stranded or double-stranded DNA molecule, a single-stranded or double-stranded RNA molecule (including messenger RNA, pre-messenger RNA and transfer RNA), an amino acid or peptide or protein bound to a ribosome and messenger RNA that codes for it (as in a ribosome display), or a peptide or protein bound to a bacteriophage and DNA that codes for it (as in a phage display), among others, alone or in some combination.

A library of such molecules is formed. The library includes one or more instances of each possible member of the k-mer of interest 112. For example, if the k-mer is 6 nucleotides at a particular location in an RNA or DNA strand, then there are 4⁶=4096 combinations of four bases taken 6 at a time and thus 4096 possible members of the k-mer. Similarly, if the k-mer is a sequence of 3 amino acids of a peptide or protein, then there are 20³=8.000 combinations of twenty amino acids taken 3 at a time and thus 8.000 possible members of the k-mer. To generate a library large enough to include multiple instances of each member of the k-mer, libraries of millions of molecules are generated in some embodiments. Any synthesizing process may be used in various embodiments.

The synthesizing process often does not produce all members at the same rate, so some members occur in a population of library molecules at a higher frequency than others. The uneven relative frequency of occurrence is illustrated on a graph, e.g. by trace 126 on a graph 120 with horizontal axis 122 that indicates individual k-mer members and vertical axis that represents relative frequency 124 (e.g., logarithm of number of occurrences in a population of 10 million molecules). The k-mer members are arranged on the horizontal axis 122 in order of decreasing frequency of occurrence. As can be seen, some members of the k-mer occur at relatively high frequency, most members of the k-mer occur in a range of intermediate relative frequencies, and some members at the far right of the trace 126 occur rarely within the library population of molecules. This distribution is a function of the synthesizing process and not a reflection necessarily of the relative frequency of occurrence of the k-mer in nature or within a natural biochemical or biological process. To obtain the relative distribution of members of the k-mer of interest, one or more Massively Parallel Sequencing (MPS) approaches are used to achieve deep sequencing of all members of the k-mer of interest and produce the trace 126. Thus, the process depicted in FIG. 1 includes sequencing the library of molecules to determine the relative frequency of each member of the k-mer in a population of library molecules.

Sequencing peptides or proteins using phage display or ribosome display is well known. See, for example, P. Dufner, L. Jermutus and R. R. Minter, “Harnessing phage and ribosome display for antibody optimization,” Trends in Biotechnology, vol. 24, 11, pp. 523-529, Sep. 4, 2006.

The population of library molecules with the known frequency distribution for k-mer members is then provided as input to a biochemical system 130, in which the k-mer will help code for a biological molecule of interest such as a functional RNA molecule, a protein, an enzyme, or supramolecular structure (e.g., a channel). In each case, a selection is imposed for the biological activity in question, such that those library members that function better are more highly represented in the output. Armed with the knowledge of how sequence determines activity, one is able to design a protein, RNA molecule or DNA molecule to suit a particular purpose. In various embodiments, selections are based on cell c survival, enzymatic activity, binding to a small or large molecule target, or any other biochemical process. In various embodiments, the library molecule is expressed by transcription or translation or some combination in a biological system, such as a cell nucleus, organelle, protoplasm, cell in vivo, or cell extract in vitro. In some embodiments, introducing the library into the biochemical system includes one or more preparation steps, such as transcribing and translating an identified nucleic acid sequence and characterizing the biological activity of the resulting protein. Thus, the method includes introducing the library of molecules into a biochemical system.

A result of one or more processes of the biochemical system 130 is a product molecule 140, at least a portion 142 of which is related to the k-mer of interest. For example, a messenger RNA molecule product 140 includes a portion 142 that was spliced from a pre-mRNA molecule transcribed from a DNA molecule 110 that includes the k-mer of interest 112. Similarly, a protein product molecule 140 output by a process of the biochemical system includes a portion 142 having amino acids that are coded by a nucleotide k-mer in an mRNA molecule 110 or related to an amino acid k-mer in a peptide or other protein. The biochemical system 130 is capable of producing a large population of product molecules. For example, the biochemical system 130 is able to output millions of product molecules to allow for the possibility of a few product molecules that include rarely occurring portions 142 related to the k-mer of interest 112.

In some embodiments, the product molecule 140 can be sequenced directly. For example, DNA can be sequenced directly. In some embodiments, a derivative molecule 150 is sequenced. The derivative molecule is both related to the product molecule 140 and sequenced for a k-mer 152 related to the portion 142 related to the k-mer of interest 112. For example, in some embodiments, the derivative molecule 150 is a complementary DNA (cDNA) molecule that is complementary to a mRNA molecule that is complementary to a portion of DNA. Since the mRNA is complementary to the original DNA, the cDNA molecule has the same sequence as the original DNA. In some embodiments, the product molecule 140 is a peptide or protein and the derivative molecule 150 is an mRNA molecule that codes for the product molecule, as determined using a bacteriophage or ribosome as in phage display and ribosome display, respectively. As used herein, an output molecule refers to either the product molecule 140 or the related derivative molecule 150, whichever is sequenced.

A large population of output molecules is sequenced to determine the relative frequency of occurrence of members of the k-mer. To adequately sample rare occurrences, millions of output molecules are sequenced using one or more Massively Parallel Sequencing (MPS) approaches to achieve deep-sequencing of all members of the k-mer of interest in the output molecules. Thus, the process includes sequencing a population of output molecules to determine the relative frequency of each member of the k-mer in a population of output molecules, wherein each output molecule is related to a product of a process of the biochemical system and each output molecule carries a k-mer related to a corresponding k-mer of a library molecule involved in the process.

The relative frequency of occurrence of members of the associated k-mer 152 is illustrated on a graph, e.g. by trace 166 on a graph 160 with horizontal axis 122 that indicates individual k-mer members and vertical axis that represents relative frequency 124 (e.g., logarithm of number of occurrences in a population of 10 million molecules). The k-mer members are arranged on the horizontal axis 122 in order of decreasing frequency of occurrence in the library population. As can be seen, some members of the associated k-mer occur at relatively high frequency, most members of the k-mer occur in a range of intermediate relative frequencies, and some members occur rarely within the population of output molecules. This distribution is a function of both the biochemical system 130 and the relative frequency of occurrence in the input population of library molecules.

To account for the effect of the uneven distribution of members of the k-mer in the library (e.g., trace 126) on the relative frequency of members of the k-mer in the output population (e.g., trace 166), each value in the output trace 166 is evaluated based on the corresponding value in the input trace 126 to determine the effect of the member within the biochemical process. For example, a ratio of values in the output trace 166 divided by the corresponding value in the input trace 126 for the same member, a, of the k-mer is computed and called the enrichment index EIa for member a. In some embodiments, a complementary sequence is transformed to the original sequence during the determination of the effectiveness. Thus the process includes determining effectiveness of each member of the k-mer based on the relative frequency of each member of the k-mer in the population of output molecules and the relative frequency of the corresponding k-mer in the library.

Because all members of the k-mer appear in the population of library molecules, the procedure described herein not only finds the members associated with high frequency in the output, which may be called enhancers of the process in the biochemical system 130 (as does SELEX, for example, albeit non-quantitatively); but also determines members that are associated with low frequencies or absence in the output, which may serve as inhibitors to one or more processes in the biochemical system 130. This positive identification of inhibitors is an advantage of a library that includes at least a few occurrences of all members of a k-mer. Such inhibitors are entirely missed by other known sequencing methods.

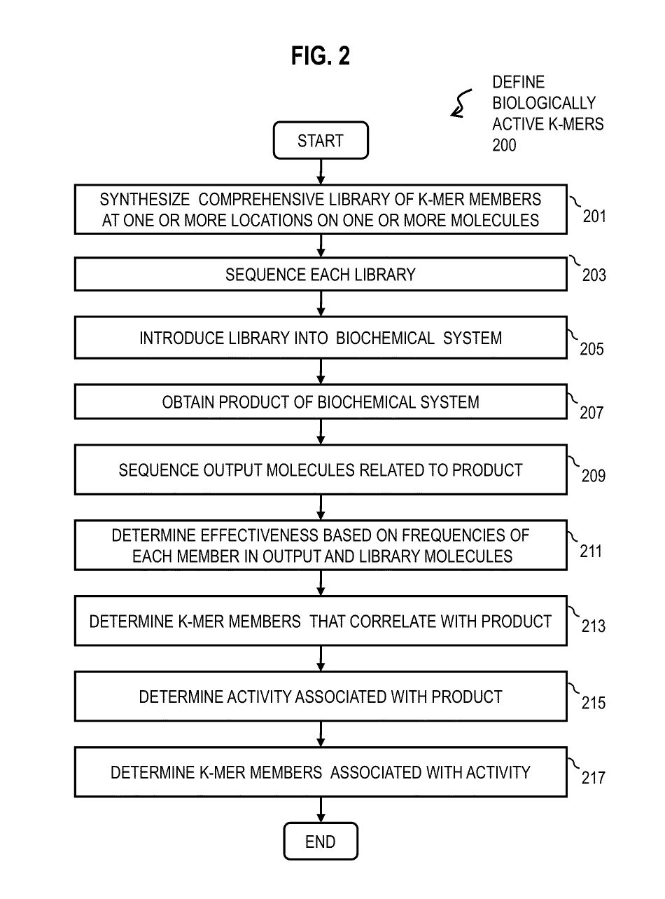

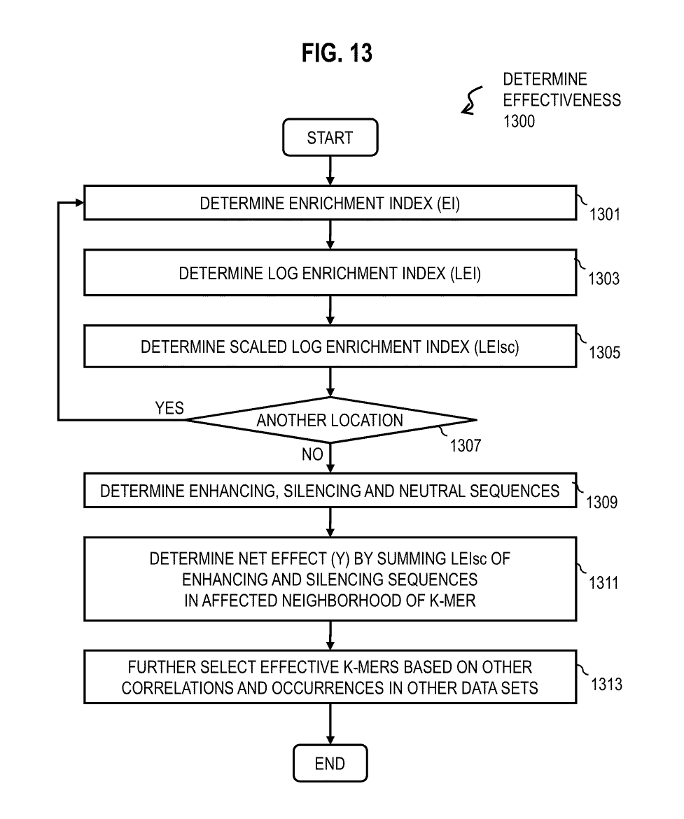

FIG. 2 is a flow diagram that illustrates an example method 200 for quantitative total definition of biologically active sequence elements, according to an embodiment. Although steps are shown in FIG. 2 (and subsequent flow diagram FIG. 13) as integral blocks in a particular order for purposes of illustration, in other embodiments one or more steps or portions thereof may be performed in a different order, or overlapping in time, in series or in parallel, or one or more steps or portions thereof may be omitted, or additional steps added, or the process may be changed in some combination of ways.

In step 201a library of molecules with comprehensive k-mer membership is synthesized. Any method may be used to generate the library, including cloning short nucleotide strands (called plasmids) in bacteria such as Escherichia coli (E. coli), or amplifying plasmids using the polymerase chain reaction (PCR), or some combination. In PCR, random members of a k-mer are obtained by amplifying two plasmid templates corresponding to regions of the library molecules adjacent to the k-mer of interest and allowing random incorporations into the PCR products.

In some embodiments the library comprises proteins or peptides. A library of proteins is produced by transferring the DNA library containing the k-mer members into a biochemical system under conditions that allow transcription and translation, such as a cell extract or in any living cell including bacterial, yeast and mammalian cells. The peptide or protein of interest is then selected by any method known in the art. One such method is based on affinity of the peptide or protein for a target molecule, e.g., in solution or attached to a solid matrix, such as a bead. In some embodiments, a cell containing the library member protein or peptide is selected on the basis of its differential survival; and then the protein or peptide or DNA or RNA that codes that protein or peptide is harvested from the selected cell. In some embodiments, a protein of interest is selected by the color or fluorescence of a product produced by the protein.

In some embodiments, it was found that E. coli did not faithfully clone some members of a k-mer. That is, upon sequencing the population of library molecules, one or more k-mer members o were missing. In such embodiments, synthesizing the library of molecules comprises synthesizing the library of molecules without using plasmids cloned in E. coli cells.

In some embodiments, PCR amplification of a limited region of a DNA template using primers with a tail harboring random k-mer members produced a large excess of sequences corresponding to those library members that happened to be complementary to the template. These offenders could be greatly reduced by using templates physically lacking the portion of the plasmid corresponding to the k-mer of interest. In some embodiments, over-representation of k-mer members corresponding to the template sequence itself was observed. In such embodiments, it was advantageous to carry out purification of templates during step 201, e.g., using a gel that contained no other nucleic acid molecules in neighboring lanes. Such an extraordinary purification step was desirable in the illustrated embodiment to eliminate contamination of the library by molecules that could diffuse from other lanes, as even in small amounts such contaminants can give rise to significant biases in the library population.

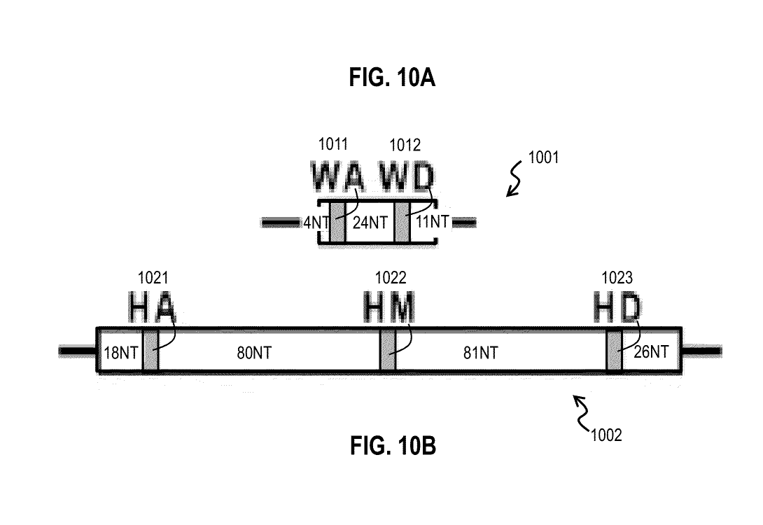

In some embodiments multiple libraries are produced during step 201. One library is produced for each of multiple contexts for inserting the k-mer, as described in more detail below with reference to FIG. 10. In such embodiments, the following steps 203 through 209 are repeated for each library.

In step 203 a population of the library molecules is deep sequenced using Massively Parallel Sequencing (MPS) approaches such as those now in wide commercial use (Illumina/Solexa, Roche/454 Pyrosequencing, and ABI SOLiD). A result of the sequencing is a trace of the relative occurrence of each member of the k-mer, such as trace 126 that is obtained if the k-mer members are sorted in order of decreasing frequency. In some embodiments, the k-mer members are sorted or plotted or both in a different order, e.g., by order 1 through b^{k} where b is the number of bases or amino acids and k in the number of positions in the k-mer. Each k-mer can be numbered from 1 to b^{k} (or from 0 to b^{k}−1) by assigning a numeric value to the bases (e.g., 0 to 3 for 4 nucleotide bases and 0-19 for the 20 amino acids) and a power to each of the k positions (e.g., k−1 to the left-most position down to 0 for the right-most position). The members of the k-mer can then be listed or plotted or both in numeric order.

In some embodiments, each frequency value is an absolute count of occurrences. In some embodiments, each frequency value is determined as the absolute count of occurrences divided by the total number of library molecules sequenced (e.g., each frequency value is a percentage less than 100% or fraction less than 1.0). The total population sequenced is large enough (e.g., multiple millions of molecules) so that even the most rare member of the k-mer is found to have multiple occurrences. Multiple occurrences for each member of a k-mer is an advantage in determining with statistical confidence which members may be inhibitors of a process in the biochemical system.

In step 205 a population of library molecules substantively identical to the population sequenced during step 203 is introduced into a biochemical system. For example, in some embodiments, a random portion of the population of library molecules synthesized during step 201 is used in the sequencing step 203; and, the remaining portion, or random subset thereof, is introduced into the biochemical system during step 205. As another example, in some embodiments, the synthesizing process generates substantively identical populations. In such embodiments the synthesizing process is used once to generate the population of library molecules sequenced during step 203; and then used again, separately, to generate the population that is introduced to the biochemical system during step 205.

In various embodiments, the biochemical system is any system of constituents and processes that are affected by the library molecules. For example, in some embodiments, the biochemical system is a cell nucleus in which a DNA strand is transcribed to a pre-mRNA strand that contains one or more introns and exons for a gene which is spliced into mRNA for the gene. In some embodiments, the biochemical system is a polyribosomal structure that assembles amino acids in a protein based on triplets of nucleotides that code for each amino acid. The code is said to be degenerate because multiple nucleotide triplets may code for the same amino acid; and, thus, a particular such amino acid may be related to any of multiple nucleotide triplets. Three nucleotides produce up to 4³=64 different codes, which are used to indicate only twenty amino acids and a stop codon. Thus some amino acids are represented by multiple codes, which provides redundancy. In some embodiments, the biochemical system is a mixture of proteins, such as in cell membranes or protoplasm, in which the presence of a protein with a particular k-mer affects the binding or folding of the same or different proteins. The system includes enough constituents to respond to each member of the library population. For example, the system includes millions of cells.

As a result of step 205 in which the library of molecules is introduced into the biochemical system, one or more processes that produce one or more molecular products are affected. Of these, one or more product molecules 140 include at least a portion 142 that is caused by, identical to, complimentary to, or otherwise related to, the k-mer 112 of interest. Example processes in various embodiments include gene transcription, mutation, gene splicing, gene activation, mRNA degradation, mRNA transport, mRNA polyadenylation, protein binding to small or large molecules (including proteins such as antibodies), protein folding, the assembly of protein complexes such as channels or signal transduction complexes, or the catalytic activity of enzymes, among others, alone or in any combination.

In step 207, one or more such product molecules that include a portion 142 related to the k-mer of interest 112 are obtained. Functional product molecules can be selectively isolated using any method known in the art. For example, in some embodiments, selection is on the basis of product moleucle size (as in spliced mRNA), hybridizability to nucleic acid molecules, affinity to small molecules such as drugs or large molecules such as proteins, or nucleic acid molecules or lipids or polysaccharides, color, fluorescence, or the ability to confer survival of a cell under prescribed conditions. These methods are presented for purpose of illustration and should not be taken to be limiting in any way. In some embodiments, the number of output products are amplified, e.g., using PCR, to obtain a sufficient sample size to sequence. In some such embodiments, the PCR outputs cDNA with an associated k-mer 152 that is the complement of the corresponding k-mer 112 of interest. In various embodiments, the output molecule is the product, e.g, mRNA or a derivative molecule, such as cDNA. In other embodiments the output molecule is a protein or other large molecule. In all cases, the output molecule is said to be related to the product molecule.

In step 209 a population of the output molecules is deep-sequenced using Massively Parallel Sequencing (MPS) approaches such as those now in wide commercial use (Illumina/Solexa, Roche/454 Pyrosequencing, and ABI SOLiD). A result of the sequencing is a trace of the relative occurrence of each member of the associated k-mer 152, such as trace 166 if the k-mer members are sorted in order of decreasing frequency in the population of library molecules. In some embodiments, the k-mer members are sorted or plotted or both in a different order, e.g., by order 1 through b^{k}.

In some embodiments, each frequency value is an absolute count of occurrences. In some embodiments, each frequency value is determined as the absolute count of occurrences divided by the total number of output molecules sequenced (e.g., each frequency value is a percentage less than 100% or fraction less than 1.0). The total population sequenced is large enough (e.g., multiple millions of molecules) so that even some rare member of the k-mer are found to have multiple occurrences. It is possible that some members of the associated k-mer are not found among the output molecules and have an absolute and relative frequency of zero. Such members may be inhibitors of the process in the biochemical system.

In step 211 the effectiveness of each member of the k-mer of interest in the process of the biochemical system is determined based on the frequency of the member in the population of output molecules and the frequency of the corresponding member in the population of library molecules. In some embodiments, the corresponding member has an identical sequence in the output and library molecules. In some embodiments, the corresponding member has complimentary sequences in the output and library molecules.

For example, in some embodiments an enrichment index (EI) is computed for each member as a ratio of the relative frequency of the member in the population of output molecules divided by the relative frequency of the corresponding member in the population of library molecules. In some embodiments, other measures are determined, such as the difference in relative frequency in the two populations. In some embodiments, the ratio of the absolute occurrences in the two populations is determined, which includes any changes of totals in the output population versus the library population. In other embodiments, the numerical data can be used as variables in equations used for a mathematical model of a process.

In other embodiments, other steps are included in step 211 to determine the k-mers that are effective in multiple contexts, as described in more detail below with reference to FIG. 13.

In step 213 the members that correlate with the product molecules are determined. For example, the members of the k-mer that are found at higher frequency in the output population than in the library population may be correlated with the product.

In step 215, an activity associated with the product is determined. For example, in some embodiments, the activity of enhanced splicing is associated with a particular gene product (e.g., a gene with three exons rather than two, as described in more detail below). As another example, in some embodiments, the activity of protein binding is associated with some product proteins.

In step 217, the k-mer members associated with the activity are determined. For example, the k-mer members highly correlated with genes that express three exons are associated with enhanced splicing. Similarly, k-mer members associated with bound proteins are associated with protein binding.

Several prior methods exist for isolating the most effective molecules in a population that carry out a particular biochemical process. SELEX (Systematic Evolution of Ligands by Exponential Enrichment) is an especially powerful example of such a process, as it is able to find the few very most effective nucleic acid molecules that carry this biological information. Although powerful, SELEX is limited in that it provides information only about the very most effective molecules, selected through multiple iterations of a selection process. That is, the output molecules are few and no information regarding their effectiveness is learned. In the method 200 presented here, information regarding the effectiveness of each member of a large population of starting molecules is obtained. The richness of this information may provide the basis for a more efficient and effective rationale design of molecules for biotechnological purposes. We call method 200 Quantodecoding for “quantitative total definition of coding information governing a biochemical process.”

### 3. Example Embodiment

In the nucleus of cells, a DNA sequence transcribed to a pre-mRNA strand includes portions (exons) that are expressed in mRNA and portions (introns) that are not. In pre-mRNA splicing, an mRNA strand is formed that excludes the introns and includes the exons of each gene. The mRNA is then translated into a peptide or protein based on codes of three nucleotides for each of 20 amino acids. In some instances, mutations occur in which one or more exons are omitted from the mRNA. It is believed that some particular nucleotide sequences, alone or in combination with other sequences, may control the efficiency of splicing in including or excluding exons. In the following embodiment, the sequences associated with enhanced and inhibited inclusion of a particular exon are determined.

Thus, in this example embodiment, a comprehensive and quantitative measure of the splicing impact of a complete set of short RNA sequences at a particular location on a pre-mRNA strand are determined using method 200. The method 200 was used to form a library with all 4096 nucleotide 6-mers at a defined position within a poorly spliced internal exon in a 3-exon minigene. A population of library DNA molecules including the minigene was sequenced; and a large population of the library molecules was transfected into cultured human cells. Millions of successfully spliced transcripts (output molecules) were then sequenced. The results provided a total list of 6-mer members that can act either as exonic splicing enhancers or silencers (ESEseqs and ESSseqs, respectively), with a digital readout of their relative strengths. These measurements were validated by RT-PCR. ESEseqs are enriched, and ESSseqs are avoided, in documented human spliced exons. Using the entire spectrum of 4096 splicing scores, correlations of high scores with exons and low scores with introns were observed. These scores also accurately predicted the effect of mutation on splicing.

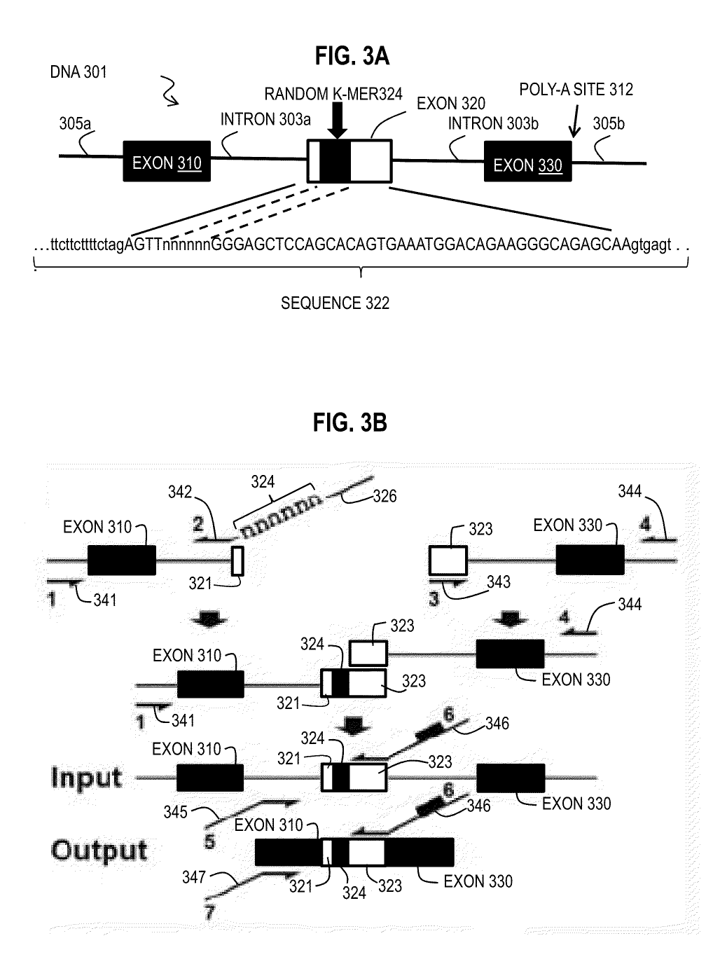

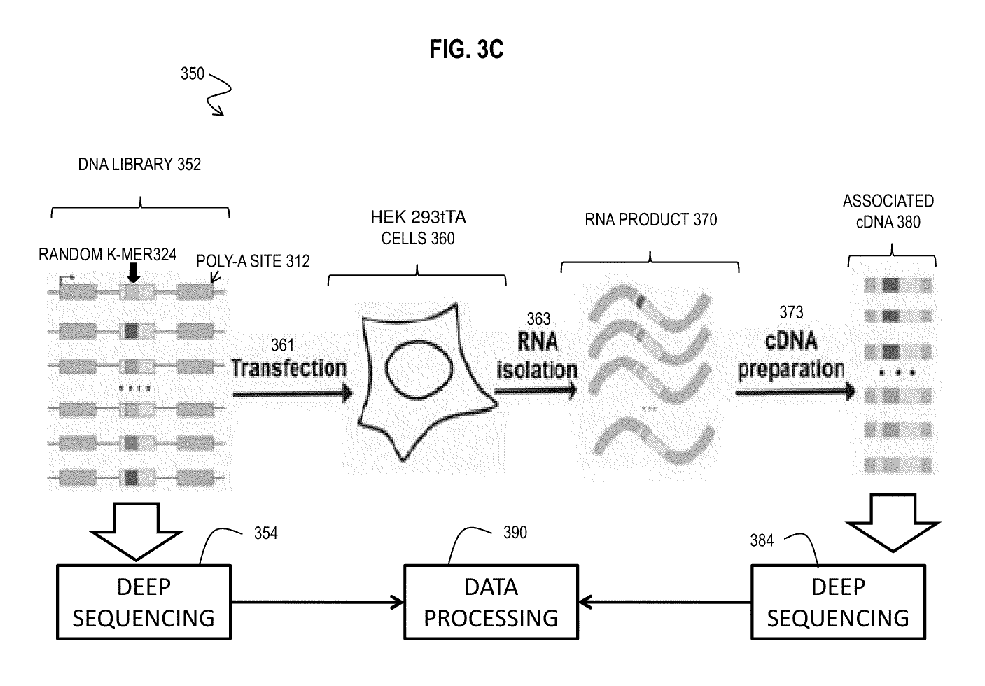

FIG. 3A is a diagram that illustrates a DNA molecule 301 of a population of library molecules used as input to a gene splicing process, according to an embodiment. The DNA molecule 301 constitutes a minigene and includes a promoter 305a and a downstream intergenic region 305b bracketing three exons 310, 320 and 330 separated by two introns 303a and 303b (collectively referenced hereinafter as introns 303). A k-mer of interest filled with random sequences for the library of molecules is indicated by random k-mer 324. In this embodiment, k=6. The third exon ends at a polyA site 312. A sequence 322 indicates the nucleotides in the vicinity of the middle exon 320. Nucleotides in the introns are lower case and in the exon 320 in upper case. The positions from 5 to 10 in the exon constitute the 6-mer of interest and are represented by the lower case letter n to indicate any of the bases may occupy any of those 6 locations.

The minigene 301 includes a tet-off promoter 305a, exon 310 of the hamster dihydrofolate reductase (dhfr) enzyme gene mutated to contain no start codons, an intron 303a derived from dhfr intron 1 and intron 303b which is an abbreviated form of dhfr intron 3, a second exon 320 derived from the human Wilms' tumor gene 1 exon 5, and a third exon 330 made up of merged dhfr exons 4 to 6 terminated by the SV40 late polyA site 312 and upstream sequence 305b. This plasmid was constructed by Mauricio Arias using standard recombinant DNA and site-directed mutagenesis methods known in the art (e.g., Molecular Cloning: A Laboratory Manual, Third Edition, J. Sambrook and David W. Russell, Cold Spring Harbor Press, Cold Spring Harbor, N.Y., USA, 2001.) The expression of this minigene requires the tTA transcription activator protein, which is provided by transfecting HEK 293tTA cells carrying an integrated copy of this gene. HEK 293tTA cells were created by Mauricio Arias by transfecting HEK 293 cells with a mammalian expression plasmid carrying the tTA gene exactly as described by Gossen and Bujard (Gossen M and Bujard H., Proc Natl Acad Sci USA. 1992, 89:5547-51). A comparable cell line (T-Rex 293) that can be used for nucleic acid/minigene expression is available commercially from Invitrogen, Life Technologies Corporation. In embodiments where transfection of a host cell is selected as the biochemical system for expression of the nucleic acid containing the k-mer of interest, any suitable plasmid that is compatible with expression in the chosen host cell can be used and engineered using any method known in the art.

The Wilms' tumor gene 1 exon 5 (WT1-5) was chosen as the central exon 320 that carries the random 6-mer library located from positions+5 to +10. The WT1-5 exon 320 was chosen because a point mutation in a predicted exon splicing enhancer (ESE) located at +6 was known to decrease exon inclusion from 100% to 4%. Thus, it was hypothesized that sequences placed at this location would be effective in modifying splicing. In addition, since this exon is only 51 nucleotides long, any stop codon in the random library will be at most 48 nucleotides from the 3′ end of the exon 320, a distance that precludes nonsense mediated decay (NMD) in most cases. The WT1-5 exon 320 also carries a T to A mutation at position+23 that was formerly inserted for past cloning experiments.

FIG. 3B is a diagram that illustrates example synthesis of the DNA molecule 301 of a population of library molecules in relation to an example cDNA molecule complementary to a spliced messenger RNA output molecule that results from a splicing process, according to an embodiment. The first fragment of the library is provided by a template including promoter 305a and intron 303a and exon 310 with a length of approximately one thousand nucleotides. The first fragment was amplified by PCR with primer 341 (SEQ ID NO. 4) and primer 342 (SEQ ID NO. 5). Primer 341 includes the nucleotides of the upstream promoter 305a. Primer 342 includes the last nucleotides of the intron 303a, the first four nucleotides 321 of the central exon 320, the random 6-mer 324, and the remaining nucleotides 326 of the central exon 320. During this step, to avoid a bias due to hybridization of the random library to the template, a PCR template that physically stops at nucleotides 321, which is short of the target 6-mer region, was used. Without this precaution, a large numbers of sequences corresponding to the template would appear in the library. The 4096 different primers 342 that span the comprehensive set of members of the random 6-mer 324 are commercially synthesized by including a mixture of all four nucleotide precursors at each of the 6 positions in successive synthesis steps.

The second fragment of the library is provided by a template including nucleotides 323 of exon 320 after the 6-mer, and intron 303b, exon 330 and downstream region 305b with a length of approximately two thousand nucleotides. The second fragment was amplified by PCR using primers 343 (SEQ ID NO. 6) and 344 (SEQ ID NO. 7). Each fragment was gel purified separately in a solitary lane of a gel chamber with no other nucleic acid molecules applied. The full-length three thousand nucleotide minigene library was generated by a subsequent overlapping PCR step using primers 341 and 344 and the first and second fragments as templates simultaneously. A mixture of RedTaq ReadyMix (Sigma) and Native Pfu DNA polymerase (Stratagene) was used for PCR. SEQ ID NO.s are collected in Table 2. Synthesizing the library of molecules further comprises using a strong promoter, such as a human cytomegalovirus (CMV) promoter.

TABLE 2
Sequence Listing

| SEQ | |
|---|---|
| ID NO. | Sequence |
| 1 | AGAGTCTGAGATGGCCTGGCT |
| 2 | GTCAGATCCGCCTCCGCGTA |
| 3 | GTAAACGGAACTGCCTCCAA |
| 4 | TGCCACCTGACGTCTAAGAA |
| 5 | CCATTTCACTGTGCTGGAGCTCCCNNNNNNAACTCTAGAA |
| | AAGAAGAAGAGGTGGGGAGT |
| 6 | GCTCCAGCACAGTGAAATGG |
| 7 | CTCCTGAAAATCTCGCCAAG |
| 8 | CAAGCAGAAGACGGCATACGAGCTCTTCCGATCTtctagct |
| | gggagcaaagtcc |
| 9 | AATGATACGGCGACCACCGAGATCTACACTCTTTCCCTACA |
| | CGACGCTCTTCCGATCT(CT or AG)TTCACTGAGCTG |
| | GAGCTC |
| 10 | CAAGCAGAAGACGGCATACGAGCTCTTCCGATCTACCGATC |
| | CAGCCTCcgcgta |

The products were then gel-purified to get rid of the templates and primers; and this completes step 201. The resulting molecules constitute the library of (input) DNA minigene molecules.

When this minigene is successfully spliced, exons 310, 320 and 330 without introns 303 are included in the population of output molecules. The middle exon includes sequence 321, random k-mer 324 and sequence 323. The output is amplified using primers 347 (SEQ ID NO. 10) and 346 (SEQ ID NO. 9) as described in more detail below.

FIG. 3C is a diagram that illustrates an example process 350 for quantitative total definition of gene splicing active sequence elements, according to an embodiment. The DNA minigene library 352 includes multiple instances of each member of the random k-mer 324, where k=6 in the middle of three exons that terminate at polyA site 312. The steps of FIG. 2 map to the processes depicted in FIG. 3C, as summarized here and described in more detail below. A first population of library molecules 352 is deep sequenced in a deep sequencing process 354 during step 203. A second population of the library molecules 352 is also transfected 361 during step 205 into a large number of living HEK 293tTA cells 360 in culture under conditions that permit the transcription of the minigene. In the transfected cells 360, the DNA library is transcribed into pre-mRNA with a complementary sequence and spliced into mRNA that retains the complementary sequence. RNA isolation 363 is accomplished during step 207 to provide a population of mRNA product molecules 370 with complementary k-mer members in those mRNA molecules that include the middle gene. In step 209, to sequence the output molecules related to the product molecules, cDNA preparation 373 converts the mRNA sequences to associated cDNA molecules 380 with sequences identical to corresponding members in the DNA library 352, though with different relative frequencies, e.g., some library k-mer members are absent in the population of output molecules. Step 209 includes sequencing a population of the associated cDNA 380 in deep sequencing process 384. In some embodiments, processes 384 and 354 are performed simultaneously. The sequences are compared and the effectiveness of k-mer members in the processes of cells 360 are inferred in data processing 390 that constitutes one or more of steps 211 through 217.

In step 203, a population of the library molecules was sequenced to determine the relative frequency of each member of the library. Step 203 includes PCR amplification and then deep sequencing. It is assumed that any PCR biases apply equally to the library and output populations, so that relative frequencies can be compared directly.

For the PCR amplification of the DNA minigene library 352, the template was the linear minigene DNA library suspended in elution buffer (EB). This library is substantively identical to the DNA library used for in vivo transfection, described in more detail below. The upstream (3′ to 5′) primer 345 (SEQ ID NO. 8) in FIG. 3B includes the standard Illumina adapter sequence followed by a sequence complementary to positions −119 to −100 in dhfr intron 1, the intron 303a upstream of exon 320. The downstream (5′ to 3′) primer 346 includes the Illumina adapter sequence, the Illumina sequencing primer template, a CG or TA barcode tag and a sequence corresponding to positions+30 to +11 in WT1 exon 5 of middle exon 320. Two separate primers with the distinct barcodes (cg or ta) were used to amplify the DNA input library in two separate experiments, to produce two duplicate samples of this library. These two populations were used to demonstrate that the amplification procedure produces substantively identical populations. Note that no ligations were necessary in this scheme, as primers specific to the constant regions of the genes being analyzed were used.

Step 203 includes deep sequencing of a population of library molecules. The PCR products of the DNA input library with distinct barcodes (cg and ta) were mixed and sequenced in a single lane on an Illumina GA II. The standard sequencing primer starts DNA synthesis at the 2 nucleotide barcode and proceeds through a 20 nucleotide upstream constant region, the 6 nucleotide random library region and an 8 nucleotide downstream constant region, for a total sequencing length of 36 nucleotides. DNA samples were quantified by fluorescence using an Agilent 2100 Bioanalyzer.

High quality 6-mers of the library were obtained by subjecting the raw sequence reads to three filters. The first filter was a sequence check for the 2 nucleotide barcode; only sequences with either a TA or CG were allowed. The second filter was a sequence check of the nucleotides upstream and 8 nucleotides downstream constant regions; only sequences with perfect matches to both were kept. The third filter was a quality check of the library 6-mer estimated from the Illumina sequence quality code provided in the raw sequencing output (probability of a correct read); the product of the quality scores for the six positions had to be at least 0.9. About half of the total reads passed all three filters. The DNA input library yielded 3,657,452 qualified 6-mer members; the qualified reads for the TA and CG barcodes were 1,827,226 and 1,830,226, respectively. In the DNA input library, the minimum count for a 6-mer member was 2 and the maximum and median counts were 2765 and 890 respectively. So the DNA input library 352 covers all 4096 6-mer members.

In step 205 a population of the library was used for the transient transfection 361 of HEK 293tTA cells 360. HEK 293tTA cells cultured in two 100 mm dishes per independent transfection (˜4×10⁶ cells total), were transfected with 2.5 micrograms (μg, 1 μg=10⁻⁶ grams) of the minigene DNA library per 100 mm dish, using Lipofectamine 2000 (Invitrogen) following the manufacturer's protocol. It was found to be desirable to transfect a relatively large number of cells and to use a strong promoter (CMV-based) to ensure a yield of purified RNA molecules sufficient to cover all members of the k-mer.

In step 207 product mRNA molecules are obtained. After cells were incubated for 24 hours, total RNA was extracted and purified using illustra RNAspin Mini Kits (GE Healthcare). A sample of 2 μg of RNA was reverse transcribed (RT) to cDNA as the output molecules using Omniscript (Qiagen) and a specific primer, AGAGTCTGAGATGGCCTGGCT (SEQ ID NO. 1), that pairs with a region in the third exon 330. RT product (cDNA) comprising 40 micro liters 1 μl=10⁻⁶ liters), which is 80% of the total RT product, was used as the template in the following PCR amplification using the same enzyme mixture mentioned above, wherein the forward primer is GTCAGATCCGCCTCCGCGTA (SEQ ID NO. 2) targeting a region near the start of exon 310. The reverse primer is GTAAACGGAACTGCCTCCAA (SEQ ID NO. 3) targeting a region in the merged exon 330. The initial denaturation step was 94° for 2 minutes; subsequent denaturation was at 94° for 45 seconds; annealing was at 60° for, 1 minute; extension was at 72° for 1 minute, each for 20 cycles; followed by a final extension at 72° for, 5 minutes. Splicing products with and without the middle exon were separated in 1.8% agarose gels stained with SYBR Safe (Invitrogen). The splicing product with the middle exon 320 was identified by its size (285 nucleotides), gel-purified and re-suspended in Qiagen elution buffer (EB).

In step 209 the cDNA output molecules derived from the mRNA product moleucles are sequenced using PCR amplification and deep sequencing. For the PCR of the population of output cDNA molecules, the template was the included splicing product suspended in EB. The downstream primer 346 was the same as for the input DNA library. The upstream primer 347 ended with a sequence corresponding to positions −105 to −86 in exon 310. Two separate primer 346 sequences with the barcodes (cg or ta) were used in amplifying the two distinct populations of the cDNA output molecules produced by independent transfections. The resulting PCR products were gel-purified to get rid of the template and PCR primers and re-suspended in Qiagen elution buffer (EB) for deep sequencing. The total size of the fragments used for sequencing was about 250 nucleotides. Note that no ligations were necessary in this scheme, as primers were used that were specific to the constant regions of the products being analyzed.

The PCR cDNA output molecules 380 of the RNA product molecules 370 with distinct barcodes (cg and ta) were pooled and sequenced similarly to the DNA library PCR products in another lane. DNA samples were quantified by fluorescence using an Agilent 2100 Bioanalyzer. High quality 6-mers of the population of output cDNA molecules were obtained by subjecting the raw sequence reads to the same three filters described above for the library. The population of output molecules yielded 3,943,635 qualified 6-mer members; the qualified reads for the ta and cg barcodes were 2,481,757 and 1,461,878, respectively. In the output cDNA molecules, the minimum count for a 6-mer members was 0 and the maximum and median counts were 8542 and 448, respectively.

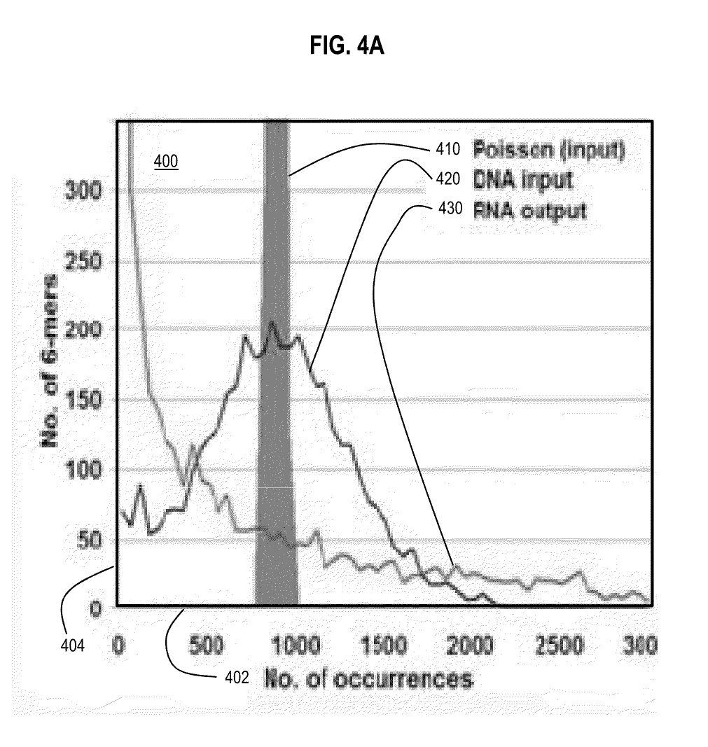

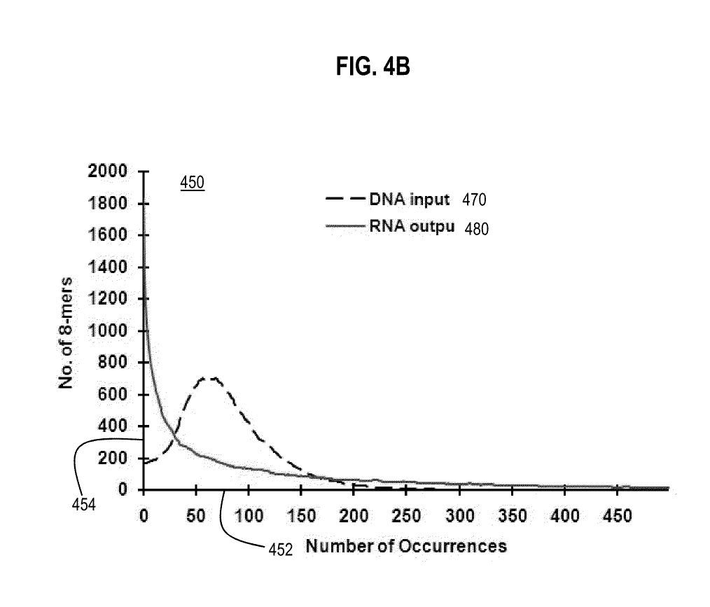

FIG. 4A is a graph 400 that illustrates an example of the relative frequency of occurrence of 4096 members of a 6-mer in a population of input library molecules and in a population of output molecules, according to an embodiment. The horizontal axis 402 indicates a number of occurrences of an individual 6-mer; and the vertical axis 404 is the number of 6-mers that had the corresponding number of occurrences. The distribution of 6-mers in the DNA input library and RNA products (as indicated by the sequencing of the output cDNA molecules) are shown as traces 420 and 430, respectively. The gray area 410 represents a Poisson distribution around the average of the input sequences. The distribution of 6-mers in the input library is wider than a Poisson distribution, suggesting that the synthesizing process does not produce a random distribution of 6-mers. The output trace 430 shows substantially more 6-mers with low occurrences (less than about 400 occurrences).

FIG. 4B is a graph 450 that illustrates an example of the relative frequency of occurrence of 65,536 members of a 8-mer in a population of input library molecules and in a population of output molecules, according to an embodiment. The horizontal axis 452 indicates a number of occurrences of an individual 8-mer; and the vertical axis 454 is the number of 8-mers that had the corresponding number of occurrences. The distribution of 8-mers in the DNA input library and RNA products (as indicated by the sequencing of the output cDNA molecules) are shown as traces 470 and 480, respectively. Distributions are similar to those depicted in FIG. 4A. This demonstrates that the method is extendable to a larger value of k.

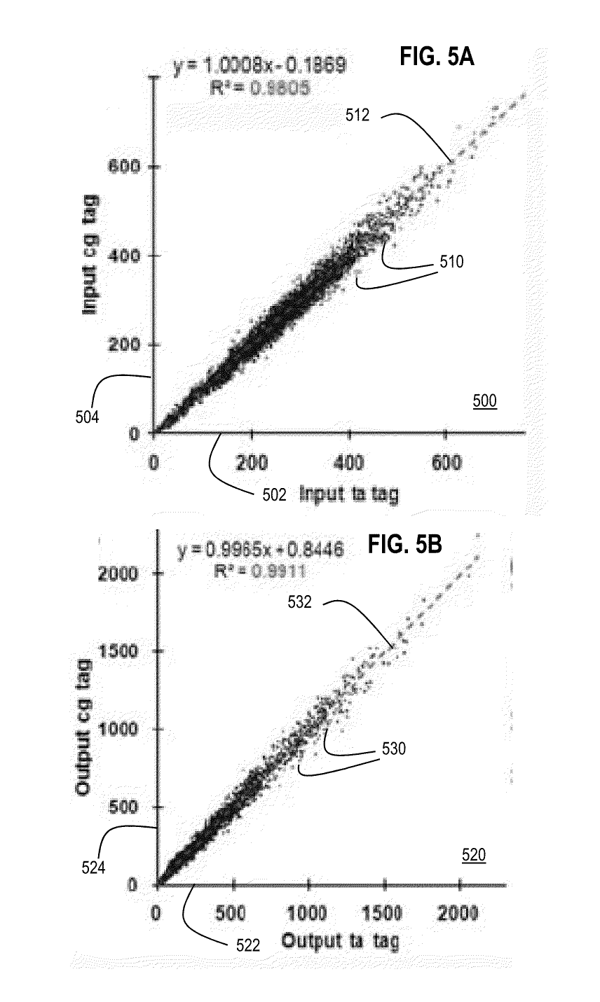

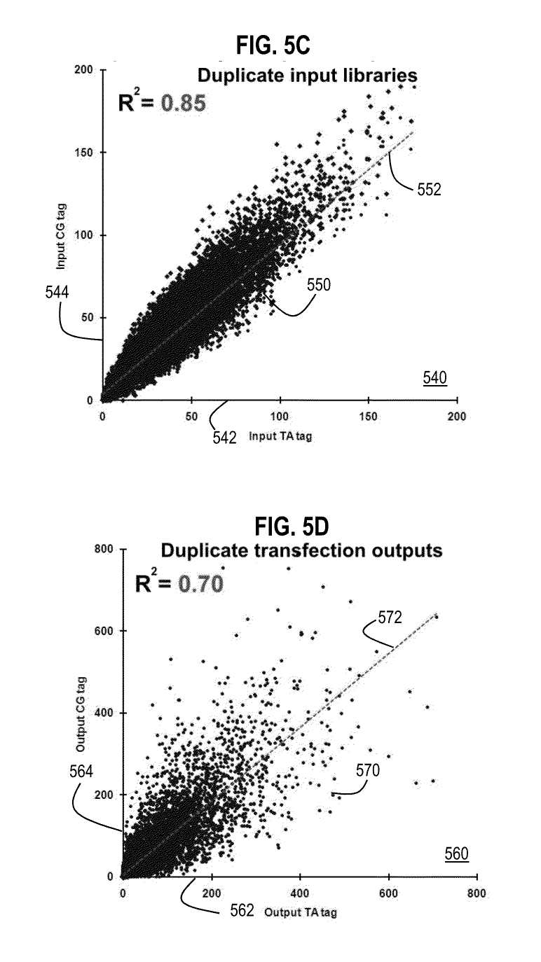

FIG. 5A is a graph that illustrates an example of the relative frequency of occurrence of 4096 members of a 6-mer in two populations of input library molecules, according to an embodiment. The horizontal axis 502 is number of occurrences per million molecules of a particular 6-mer member tagged with the two nucleotides ta in the downstream primer. The vertical axis 504 is number of occurrences per million molecules of the identical 6-mer member tagged with the two nucleotides cg in the downstream primer. The individual 6-mers indicted by dots 510 are fit by line 512. The results show R²=0.98 and a slope of 1.0. This indicates the two library populations are substantively identical.

FIG. 5B is a graph that illustrates an example of the relative frequency of occurrence of 4096 members of a 6-mer in two populations of output molecules, according to an embodiment. The horizontal axis 502 is number of occurrences per million molecules of a particular 6-mer tagged with the two nucleotides ta in the downstream primer. The vertical axis 504 is number of occurrences per million molecules of the identical 6-mer tagged with the two nucleotides cg in the downstream primer. The individual 6-mers indicted by dots 530 are fit by line 532. The results show R²=0.99 and a slope of 1.0. This indicates the two output populations, originating from two independent transfections, are substantively identical.

FIG. 5C is a graph that illustrates an example of the relative frequency of occurrence of 65,536 members of a 8-mer in two populations of input library molecules, according to an embodiment. The horizontal axis 542 is number of occurrences per million molecules of a particular 8-mer member tagged with the two nucleotides ta in the downstream primer. The vertical axis 544 is number of occurrences per million molecules of the identical 8-mer member tagged with the two nucleotides cg in the downstream primer. The individual 8-mers indicted by dots 550 are fit by line 552. The results show R²=0.85 and a slope of 1.0. This indicates the two library populations are substantively identical.

FIG. 5D is a graph that illustrates an example of the relative frequency of occurrence of 65,536 members of a 8-mer in two populations of output molecules, according to an embodiment. The horizontal axis 562 is number of occurrences per million molecules of a particular 8-mer tagged with the two nucleotides ta in the downstream primer. The vertical axis 564 is number of occurrences per million molecules of the identical 8-mer tagged with the two nucleotides cg in the downstream primer. The individual 8-mers indicted by dots 570 are fit by line 572. The results show R²=0.70 and a slope of 1.0. This indicates the two output populations, originating from two independent transfections, are substantively identical. FIG. 5C and FIG. 5D again demonstrate the method of FIG. 2 is extendable to larger values of k.

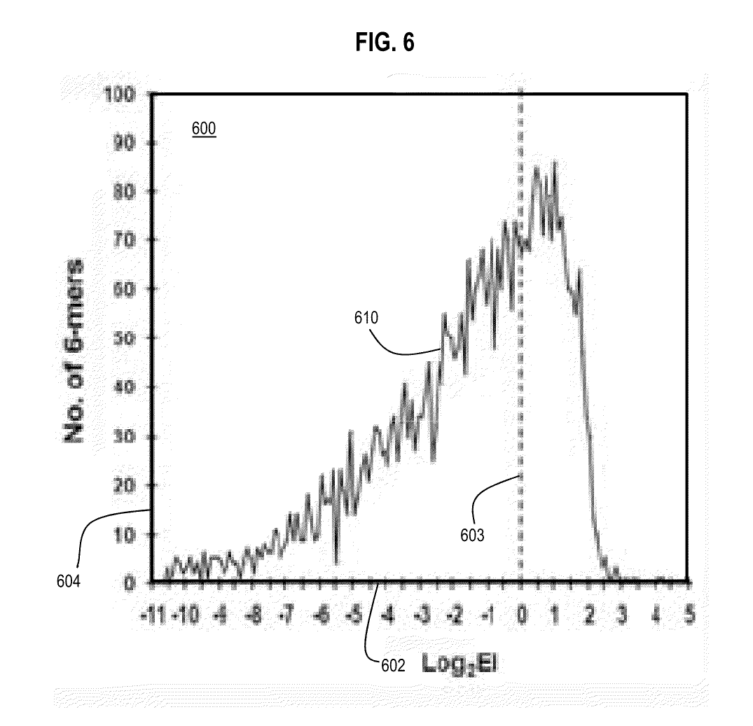

FIG. 6 is a graph 600 that illustrates an example distribution of the splicing enrichment index (EI) among 4096 members of a 6-mer, where an EI is a ratio of relative frequency of a 6-mer member in the population of output molecules that include the middle gene 320 to the relative frequency of the same 6-mer member in a population of library molecules, according to an embodiment. The horizontal axis 602 is the logarithm of EI relative to a base 2 (Log₂(EI)). The vertical axis is number of 6-mers exhibiting that EI. EI values greater than 1 indicate enhancement (higher relative occurrence in the output molecules) and have positive Log₂ values. EI values less than 1 indicate inhibition (lower relative occurrence in the output molecules) and have negative Log₂ values. Many k-mer members suffer substantial inhibition with ratios of 0.1 (Log₂ values of −3.4) and less.

Because all the 4096 6-mer members were covered in the input DNA library, an EI can be calculated for every 6-mer member during step 211. For a particular 6-mer member, called member a, its proportion of inclusion, A, in the spliced gene is equal to EIa times the overall proportion of inclusion for the whole library, L, as indicated by Equations 1a through 1e.

N=T*L (1a)

where N is the total number of molecules in the population of output molecules that include the middle exon 320, T is the total number of molecules in the population of library molecules transfected into the cells 360, and L is the overall proportion of inclusion of the middle exon for the whole library. By definition,

EIa=Oa/Ia (1b)

where Oa is the relative frequency of member a in the population of output molecules that include the middle exon, and Ia is the relative frequency of member a in the population of library (input) molecules.

Ta=Ia*T (1c)

where Ta is the number of molecules that include member a in the population of library molecules.

Ma=Ia*T*A (1d)

where Ma is the number of molecules that include member a in the population of output molecules and A is the proportion of inclusion of member a in the spliced mRNA. Thus, the relative frequency of member a in the output is

Oa=Ma/N=(Ia*T*A)/(T*L)=Ia*A/L (1e)

and

EIa=Oa/Ia=(Ia*A/L)/Ia=A/L (1f)

### Thus,

A=EIa*L (1g)

So EIa=A/L and for the illustrated embodiment. The value of L was measured to be ˜16% based on band intensities after RT-PCR. The maximum value for A is 100%. Thus the maximum value for EIa is about 1/0.16=6.25. Indeed, the EIs of most 6-mer members (99.8%) were less than 6.25. Of the ten 6-mer members that had EI values greater than 6.25, all had a relatively low number of input DNA library counts (their input counts were all much less than the median input value of all 6-mers) and so had a less reliable estimate of EI. In the population of output molecules, there were 56 total 6-mer members with 0 counts and their EI values were zero accordingly. In the transformation from EI to Log₂ EI (LEI), because Log₂(0) is infinite, a pseudo output count of 1 was assigned to these 6-mer members with a count of zero. Although 56 6-mer members have the same EI value of 0, the 6-mers with higher input proportions are likely to be stronger silencers, and accordingly resulted in lower LEI values. The LEI distribution of all 4096 6-mer members is shown in FIG. 6.

To estimate the statistical significance of enrichment or depletion in the population of output molecules compared to the DNA input library for each of the 4096 6-mer members, a modified negative binomial model (edgeR47) was used. The data from the two independent transfections and the two populations of DNA library molecules were used. The 6-mer members with EI values of greater than 1 were considered to be ESEseqs; and those with EI values less than 1 to be ESSseqs. For a 5% false discovery rate (FDR) cutoff, there are 1327 ESEseqs and 2502 ESSseqs. Thus, in this embodiment, during step 213, an EI greater than 1 is correlated with mRNA product molecules that more efficiently include the middle exon.

The division at an EI of one reflects the influence of 6-mer members relative to the average for the input library, but is of an arbitrary nature and does not necessarily reflect the mechanism by which these sequences act to govern splicing. Thus, in step 215 and 217, the effect of particular EI values on splicing is determined.

Fourteen 6-mer sequences, the Els of which cover a wide range of values, were chosen to validate the idea that their EIs reflect their quantitative splicing efficiencies. Each of the fourteen 6-mer members was cloned into the random library position of the 3 thousand nucleotide linear minigene construct. HEK 293tTA cells cultured in 35 millimeter dishes were transfected as described above, except splicing products were stained with ethidium bromide. The intensity of each splicing product was quantified with ImageJ. At least two independent transfections were performed for each construct. Proportion included (P) was defined by Equation 2.

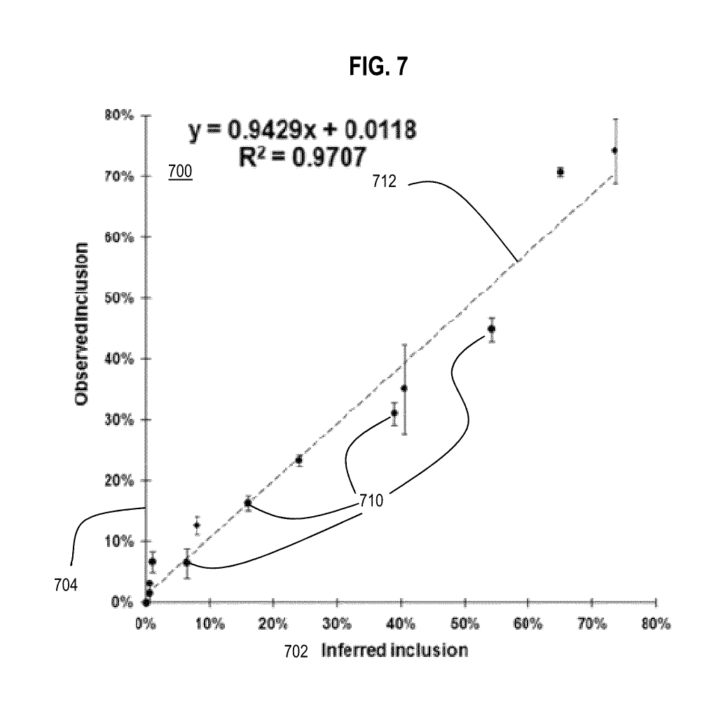

P=included product/(skipped product+included product) (2)

where skipped and included product amounts are expressed in molar quantities. FIG. 7 is a graph 700 that illustrates a relationship between a rate of inclusion of an exon in a spliced mRNA molecule based on the enrichment index EI compared to an observed rate of inclusion, according to an embodiment. The horizontal axis 702 is inferred inclusion using EI for the 6-mer member and Equation 1g. The vertical axis 704 is observed inclusion using Equation 2. The trace 712 depicts a straight line fit with slope 0.9 and R²=0.97. Graph 700 illustrates a linear relationship between an observed rate of inclusion of an exon in a spliced mRNA and a rate of inclusion of the exon based on the enrichment index EI. Thus, the observed inclusion proportions of 14 tested 6-mer members agree well with those inferred from the sequencing data.

Having identified 6-mer members that serve as splicing enhancers and inhibitors, it is possible to see their effects on other gene sequencing data to generalize the effect of the members on the splicing activity, e.g., in step 217. Such analysis is provided in a later section.

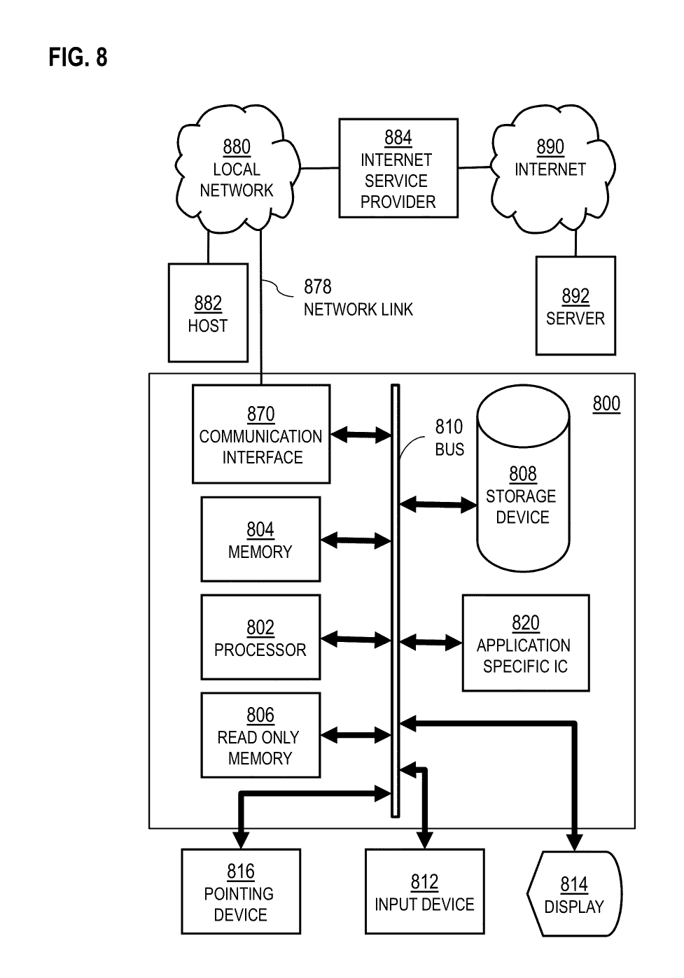

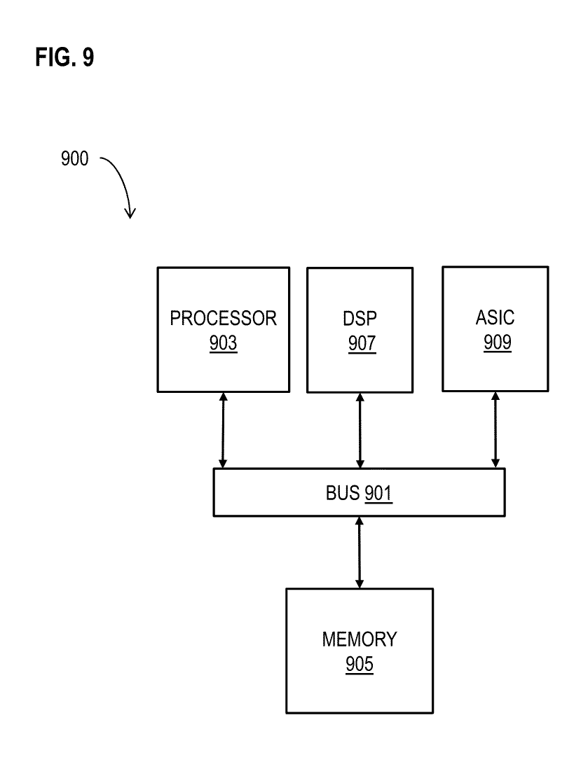

In some embodiments, one or more of steps 211 through 217 are performed using computational hardware, as described in a later section below with reference to FIG. 8 and FIG. 9.

### 4. Context Adjustments

The effect of a k-mer (motif) may depend on the sequence that surrounds the k-mer, e.g., because of the interactions those surrounding sequences induce, such as propensity to be single-stranded, interactions with remote sequences, and strength of binding with enzymes that promote certain activities, such as splicing. To account for the context of the k-mer, in various embodiments, the k-mers changed in the neighborhood of the introduced k-mer, or the location of the k-mer within a molecule, or the molecule to which the k-mer is introduced, or some combination are taken into consideration.

For example, the effect of a splicing regulatory motif can depend on the RNA sequence that surrounds it. The extent of such effects were examined in an illustrated embodiment by extending the experiment described above to test a total of five locations, as follows: WA, near the acceptor site (39 splice site) preceding the WT1-5 exon (51 nt), described above; WD, near the donor site (59 splice site) of WT1-5; HA, near the acceptor site of human beta globin exon 2 (Hb2, 223 nt); HM, near the middle of Hb2; and HD, near the donor site of Hb2. FIG. 10A and FIG. 10B are block diagrams that illustrate example different locations for each k-mer, according to an embodiment. The WT1-5 exon 1001 is depicted in FIG. 10A, along with the WA location 1011, described in the previous experiments, and the new WD location 1012. The WA location is 4 nucleotides (nt) from the 3′ end, 24 nt from the WD location 1012. The WD location is therefore 11 nt from the 5′ end of the exon. The Hb2 exon 1002 is depicted in FIG. 10B, along with the acceptor HA location 1021, the middle HM location 1022 and the donor HD location 1023. The HA location 1021 is 18 nt from the 3′ end and 80 nt from the HM location 1022. The HM location 1022 is 81 nt from the HD location 1023 that is therefore 26 nt from the 5′ end of the exon.

To compare the results from different locations, all EI scores are expressed as the log2 (LEI) so as to give comparable weight to enhancers and silencers. The LEI values from each location were scaled so that the median value is zero and the range from −1 to +1 captures 95% of the k-mers. For example, the median value is subtracted from the LEI value and the positive values are divided by the 97.5^{th} percentile value of the difference and the negative values are divided by the 2.5^{th} percentile value of the difference. This scaled LEI is abbreviated LEIsc. The LEIsc value of a k-mer represents the behavior of a molecule harboring it at a particular location in a particular molecule.

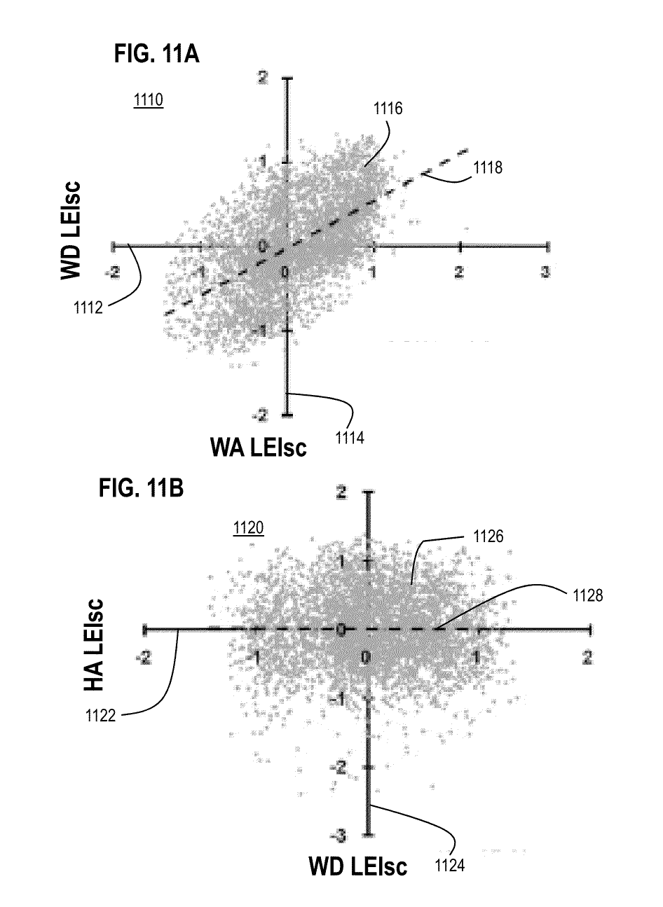

For example, the LEIsc value of a 6-mer represents the splicing behavior of a pre-mRNA molecule harboring it at a particular location in a particular exon. The 10 pairwise comparisons of LEIscs between the five locations generally showed fair to poor correlations with a median R² value of 0.10. The best (WA vs. WD) yielded an R² of 0.34. FIG. 11A is a graph 1110 that illustrates similar effectiveness of k-mers in two different locations, according to an embodiment. The horizontal axis 1112 indicates the WA LEIsc values; and, the vertical axis 1114 indicates the WD LEIsc values. The individual k-mers are represented by dots 1116 and the straight line fit by line 1118. The worst correlation (HA vs. WD) yielded a negligible R² of 3×10⁻⁵. FIG. 11B is a graph that illustrates dissimilar effectiveness of k-mers in two different locations, according to an embodiment. The horizontal axis 1122 indicates the WD LEIsc values; and, the vertical axis 1124 indicates the HA LEIsc values. The individual k-mers are represented by dots 1126 and the straight line fit by line 1128. Thus, the context of a substituted 6-mer can greatly influence its effect. Despite the variability seen between locations, LEIscs seem to be identifying ESEs and ESSs that are generally used, since 6-mers with high scores at each location were found to be enriched and 6-mers with low scores depleted in human exons compared with introns. Furthermore, the average LEIsc value of a k-mer across all locations tends to indicate consistent enhancers and silencers. It was found that exons with lower average LEIsc values taken from each location tend to have stronger 3′ and 5′ splice site sequences. LEIsc scores might be expected to compensate for weak splice sites and vice versa.

One source of difference between any two locations lies in the nature of the k−1 bases that flank each side of the site of a k-mer substitution. As these are different at each site, each of the 4^{k} substitutions gives rise to a potentially unique set of 2k−1 overlapping k-mers (from −(k−1) to +(k−1)) relative to the ends of the substitution at each location. For any particular input molecule, the dominant behavioral sequence may well lie within one or more of the overlapping k-mers in this (3k−2) nt region rather than being the substitution k-mer itself. This state of affairs could be the source of much of the apparent variation seen among different substitution locations. To take this overlap effect into account, for each possible k-mer the LEIsc values were collected from all input molecules that contained it anywhere within the (3k−2) nt region. The average of these LEIsc values was calculated and compared with the average of the LEIsc values of molecules that did not contain the k-mer. The k-mers with significantly higher averages were considered enhancers; and, the k-mers with significantly lower averages were considered silencers. A score difference was computed as the difference between the average LEIsc of the significant k-mer compared to the average LEIsc of the molecules that did not include the k-mer. For purposes of illustration it is assumed that NE is the number of k-mers found to be enhancers and NS is the number of k-mers found to be silencers.

In some embodiments, an additive model to calculate the net effect of the (2k−1) overlapping k-mers found in a given input molecule, weighting each enhancer and silencer present by its average LEIsc score. This net effect (y) is given by Equation 3.

$$y = {\sum }_{i = 1 , NE} Ei \times ai + {\sum }_{j = 1 , NS} Sj \times bj \qquad ( 3 )$$

where Ei and Sj are the enhancer average LEIsc score difference and silencer average LEIsc score difference, respectively; ai and bj are the occurrences of the corresponding k-mers within all (2k−1) overlapping k-mers; and y is the predicted behavioral strength of the input molecule. For example, as described in the next paragraphs, a predicted splicing strength was calculated using Equation 3 for each of 20,480 pre-mRNA molecules. The observed LEIsc values agreed well with these predicted values.

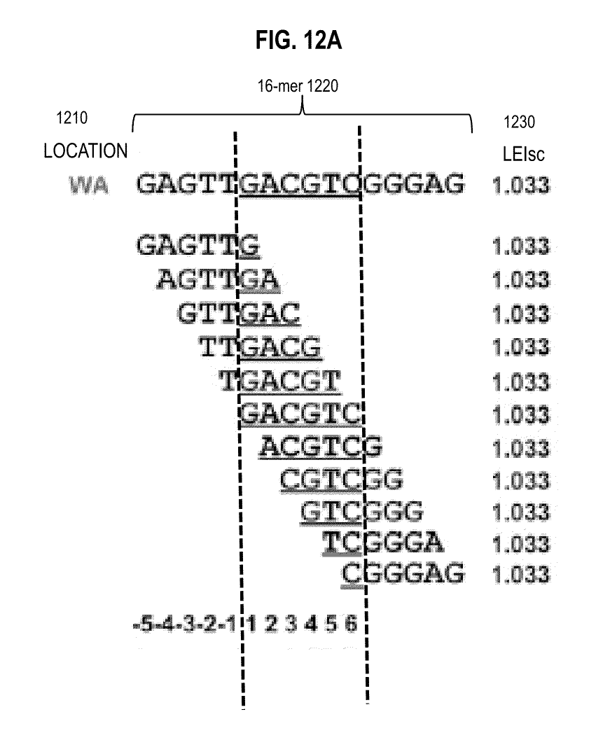

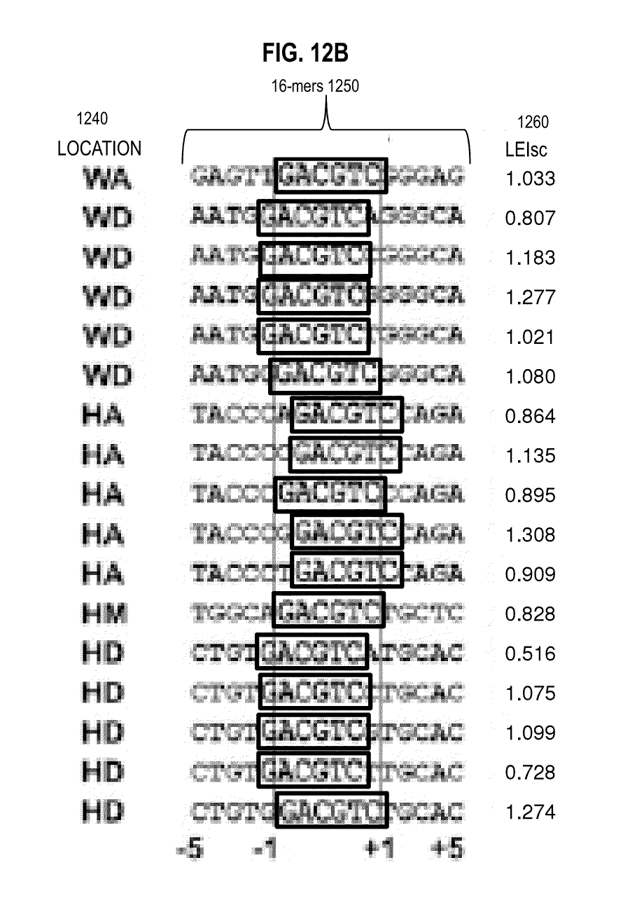

For example, one source of difference between any two locations lies in the nature of the five bases that flank the site of 6-mer substitution. As these are different at each site, each of the 4096 substitutions gives rise to a unique set of 11 overlapping 6-mers (in a 16-mer extending from −5 to +5 relative to the ends of the substitution). FIG. 12A is a diagram that illustrates example overlapping k-mers changed by substitution of one k-mer in one location, according to an embodiment. The 6-mer is substituted at the underlined positions bracketed by vertical dashed lines in the 16-mer 1220 of the WA location indicated in column 1210. In this substitution, the LEIsc was found to be 1.033, as indicated in column 1230. However, the substitution at the underlined positions creates eleven different overlapping 6-mers, using various numbers of the flanking nucleotides as indicated by the eleven rows, starting a positions −5 though +6. At a different location with different flanking nucleotides the LEIsc is often different for the same ti-mer.

The overlapping sequences are considered as 6-mers for consistency. For any particular mutant pre-mRNA molecule, the dominant splicing regulatory sequence may well lie within one or more of the overlapping 6-mers in this 16-nt region rather than being the substitution 6-mer itself. This state of affairs was found to be the source of much of the apparent variation seen among different substitution locations.

To take this overlap effect into account, for each possible 6-mer the LEIsc values were collected from all pre-mRNA molecules that contained the 6-mer anywhere within the 16-nt region. For example, the 6-mer GACGTC (SEQ. ID 11) was created 17 times among all five locations. FIG. 12B is a diagram that illustrates example multiple occurrences of one k-mer (GACGTC, SEQ. ID 11) in different locations, according to an embodiment. The location is indicated in column 1240, the 16-mer at that location by column 1250 and the LEIsc in column 1260. The GACGTC (SEQ. ID 11) motif occurred once each in the WA and HM locations and five times each in WD, HA, and HD. Each of these occurrences is associated with a particular pre-mRNA molecule and a particular LEIsc value for that molecule as indicated in column 1260. The average of these LEIsc values was calculated. A t-test was used to compare this average with the average of the LEIsc values of molecules that did not contain the 6-mer (e.g., GACGTC, SEQ. ID 11). This latter value is always close to zero since it is comprised of almost all of the 20,480 (5×4096) molecules considered. If a 6-mer had a significantly higher average LEIsc value (P<0.05, t-test) it was viewed as splicing enhancer (ESEseq,), and we defined its ESEseq score as the difference between the averages of the two categories described above (present vs. absent). ESSseq scores were defined similarly for 6-mers that had a significantly lower average LEIsc value. The term “ESRseq” refers to the above two categories as a group. The 6-mers that showed no significant differences have been provisionally regarded as neutral.

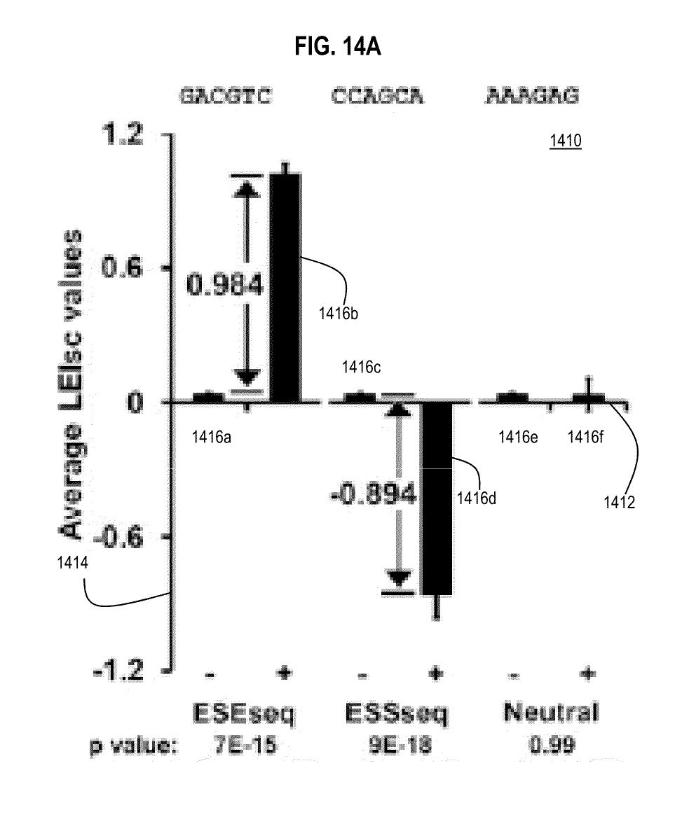

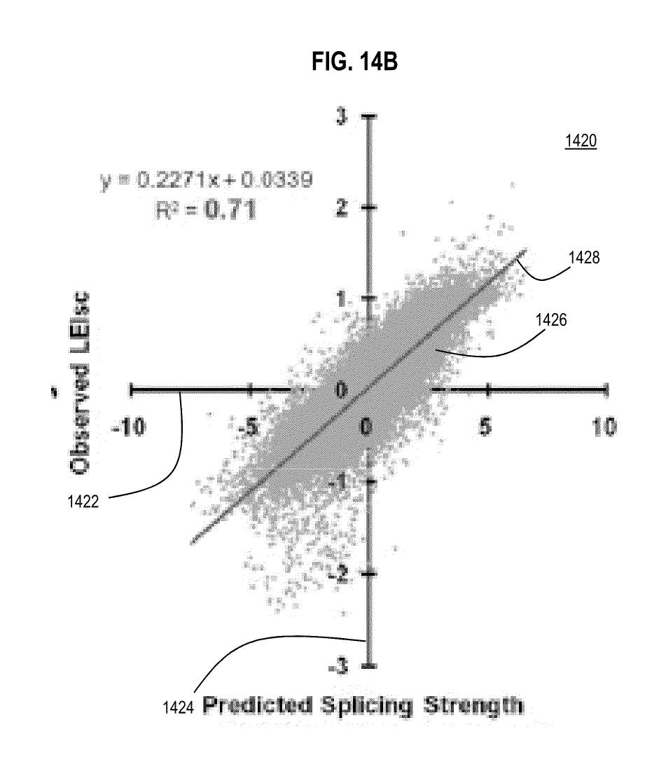

FIG. 14A is a graph 1410 that illustrates example average effectiveness scores of enhancing sequences, silencing sequences and neutral sequences, according to a splicing embodiment. The vertical axis 1414 indicates the average LEIsc values, the horizontal axis 1412 indicates a particular 6-mer. Three example 6-mers are shown, a signifcantly enhancing 6-mer, a significantly silencing 6-mer, and a neutral 6-mer. For each 6-mer the average LEIsc for input molecules that include the 6-mer is shown in a + column (present) and the average LEIsc for input molecules that do not include the 6-mer is shown in a − column (absent). The average LEIsc 1416a for input molecules absent GACGTC (SEQ. ID 11) is near zero and the average LEIsc 1416b for input molecules with GACGTC (SEQ. ID 11) present is 0.984 greater, significant at p=7×10⁻¹⁵, indicative of a significant enhancing 6-mer. The average LEIsc 1416c for input molecules absent CCAGCA (SEQ. ID 12) is near zero and the average LEIsc 1416d for input molecules with CCAGCA (SEQ. ID 12) present is 0.894 less, significant at p=9×10⁻¹⁸, indicative of a significant silencing 6-mer. The average LEIsc 1416e for input molecules absent AAAGAG (SEQ. ID 13) is near zero and the average LEIsc 1416f for input molecules with AAAGAG (SEQ. ID 13) present is about the same, p=0.99 likely to be the same distribution, indicative of a neutral 6-mer.

Failure to achieve a significant difference depends on two factors: the variance among the results from the five different locations and the magnitude of the effect on splicing. In this way, we defined NE=1182 ESEseqs (FDR=17.3%) and NS=1090 ESS seqs (FDR=18.8%) as well as their ESRseq scores. Similar results were obtained using a Kolmogorov-Smirnov (K-S) test. A few 6-mers appear more than once in an overlap region. In these cases we counted only the presence or absence of the 6-mer, as a regression model in which the effect on splicing was assumed to be linearly dependent on the number of occurrences of these 6-mers produced virtually the same results

FIG. 14B is a graph that illustrates example relationship between LEIsc values and predicted effectiveness, according to a splicing embodiment. The horizontal axis 1422 is predicted splicing strength (not averaged); and the vertical axis 1424 is observed LEIsc. The graph 1420 compares the observed LEIsc value of a library pre-mRNA molecule with the splicing strength (y) predicted from the additive model of Equation 3. The chart contains 20,480 points 1426 (4096 6-mers times 5 locations) and shows about 30% variability (R²=0.71) with a straight line fit 1428. The R² values for each individual location ranged from 0.53 to 0.84.

The additive model was also tested by leaving out one location and using the remaining four for prediction; the predictions for the left-out location were then tested against the corresponding observed LEIsc values. The observed LEIsc values again agreed well with the predicted values, with R² values ranging from 0.21 to 0.67 for the five tests and 0.39 overall. It is concluded that the additive model successfully takes into account the contributions of the created overlapping sequences, and that such sequences are responsible for a large part of the context effect. The overlap effects explain 70% of the variance in observed splicing behavior. The remaining 30% is likely due to context effects other than overlaps such as proximity to a splice site, secondary structure, and combination effects. Additional sources of context effects are considered below.

FIG. 13 is a flow diagram that illustrates an example method 1300 for determining context adjusted effectiveness of biologically active sequence elements, according to an embodiment. Method 1300 is a specific embodiment of steps 211 to 217 depicted in FIG. 2.

In step 1301, an enrichment index (EI) is determined, e.g., according to Equation 1b, described above, for each k-mer in the comprehensive library. In step 1303, the log EI is determined, e.g., log₂ (EI). In step 1305, a scaled enrichment index is determined, e.g., by subtracting the median value and dividing the positive differences by the 97.5 percentile difference value and dividing the negative values by the absolute value of the 2.5 percentile difference value.

In step 1307, it is determined if there is another location for which input library sequences and product sequences are available. If so, control passes back to step 1301 to repeat steps 1201, 1303 and 1305 for the next location. If not, control passes to step 1309.

In step 1309, significant enhancers, silencers (or inhibitors) and neutral k-mers are determined. For example, the distribution of LEIsc values is determined for input molecules in which the k-mer is present anywhere in the overlapping k-mers at each location and compared to the distribution of LEIsc values for input molecules in which the k-mer is absent. The k-mers having distributions with significantly higher LEIsc values when present than when absent, e.g., significantly higher average values, are considered enhancing sequences. The k-mers having distributions with significantly lower LEIsc values when present than when absent, e.g., significantly lower average values, are considered silencing or inhibiting sequences. The k-mers having distributions with insignificant differences in LEIsc values when present than when absent are considered neutral sequences. In some embodiments, step 1309 is a specific embodiment of steps 213 and 215.

In step 1311, the net effect of a substitution of a k-mer at a particular location is determined based on the occurrence of enhancing and silencing sequences. For example, the value y is determined as given by Equation 3, described above. In some embodiments, step 1311 is a specific embodiment of step 217.

In step 1313, the enhancing or silencing sequences, or both, are further refined and selected based on other correlations or occurrences in other data sets, or some combination. Examples of use of such other data sets are described in the next section. In some embodiments, step 1313 includes determining the context effects other than overlaps such as proximity to a splice site, secondary structure, and combination effects.

Nonsense mediated decay (NMD). In some locations, some k-mer substitutions could give rise to in-frame premature termination codons (PTC) at the substitution location if an ATG triplet in a central exon is used as a start site. The possibility was considered that some poor representation of mRNA molecules was due to nonsense-mediated decay (NMD) rather than inefficient splicing. At the WA, WD, and HD locations, these PTCs will reside at positions <50 nt from the end of a penultimate exon, positions from which NMD is not usually seen. Such is not the case for locations HA and HM. Evidence of an NMD bias in the Enrichment Index was examined for these locations. An examination of trinucleotide normalized frequencies showed the stop codons TAA and TAG were among the lowest. However, NMD is unlikely to be the cause, as this result was also seen at locations that should be immune to NMD (WA, WD, and HD), and the low frequencies were not sensitive to position within the exon (potential reading frame). Most telling, the TGA stop codon in all three reading frames at all five locations is not selected against, occurring with a frequency close to the average (1.56%, 1/64).

Positional bias. Splicing regulatory factors (e.g., SR proteins and hnRNPs) may participate differentially in the recognition of 3′SSs and 5′SSs. Such selectivity could give rise to a positional bias for proximity to one or the other splice site. Such specificity was examined by extracting 6-mers that exhibited differential effects, depending on whether they were close to the 3′SS (HA location) or close to the 5′SS (HD location) in the long (223 nt) Hb2 exon.

HA context preferred motifs are more highly enriched in the exonic region closer to the 3′SS in human constitutive exons. HD context preferred motifs are more highly enriched in the exonic region closer to the 5′SS. HD context preferred motifs resembling 9G8 binding sites are more highly enriched in the exonic region closer to the 5′SS in human constitutive exons. HD context preferred motifs resembling PTB binding sites are less depleted in the exonic region closer to the 5′SS.

When a library was placed at the WD location, a minor (10%) use of a downstream (“proximal” relative to the intron) cryptic 5′SS was noticed. Sequencing this minor class of molecules allowed the definition of 6-mers that tended to either enhance or silence the use of the cryptic site. Six-mers that exhibited a significantly higher use of the wild-type 5′SS were found to be enriched in the region upstream of the 5′SS in human constitutive exons (defined below). Accordingly, 6-mers that exhibited a lower use of the wild-type 5′SS were found to be depleted in this region. The latter could be a candidate for silencers that encourage the use of an alternative splice site.

RNA secondary structure (single vs. double stranded). RNA secondary structure has been shown to influence splicing in many individual cases and may act in general by keeping many splicing elements single stranded to allow the binding of protein factors. In support of this idea the literature reports that predicted ESE sequences in human exons tend to remain single stranded.

Embodiments of the present invention provide an unprecedented opportunity to tie observed splicing efficiencies to computationally calculated secondary structures in thousands of RNA molecules that differ only in a prescribed k-mer region. The method of Hiller M, Zhang Z, Backofen R, Stamm S., “Pre-mRNA secondary structures influence exon recognition,” PLoS Genet. 3: e204. doi: 10.1371/journal.pgen.0030204 (2007), the entire contents of which are herby incorporated by reference as if fully set forth herein, was applied to calculate the predicted single-stranded state of ESRseqs in all five locations. As applied, the method comprised calculating the predicted folding free energy of 20 windows of increasing size (28-66 nt) centered on a k-mer. Folding was calculated allowing or disallowing pairing of the 6-mer bases and the energy differences were converted to pairing probabilities (PU, the probability of being unpaired). The average of the 20 PU values was assigned to each k-mer.

It was asked whether ESEseqs that promote the splicing of a transcript are found in regions of different secondary structure than ESEseqs that do not. We compared two sets of ESEseqs: set 1, all ESEseqs residing in transcripts with high LEIsc values (top 400) and set 2, all ESEseqs residing in transcripts drawn from those with average LEIsc values (middle 1000). These ESEseqs could be located anywhere within the 16-nt region defined by positions overlapping the substituted 6-mer.

Because G+C content is a major determinant of RNA secondary structure, these two sets were matched for G+C content at two levels. First, on a one-to-one basis, each 6-mer substitution in set 2 was chosen so as to match the G+C content of a 6-mer substitution in set 1. Second, on a one-to-one basis, each ESEseq in set 2 had to match the G+C content of an ESEseq in set 1. In this way both sets contained the same distribution of molecules with respect to G+C content in the region being locally folded. PU values were then calculated for each set; each of the five substitution locations was analyzed separately (e.g., the matching took place only within a location). In each case, the mean PU of set 2 was set equal to unity for comparison. The actual PUs for ESEseqs in set 2 were: 0.037 for WA, 0.075 for WD, 0.057 for HA, 0.099 for HM, and 0.062 for HD.

To ask whether ESSseqs that silence splicing are found in regions of different secondary structure from ESSseqs that do not, two sets of ESSseqs were compared, exactly as described above for ESEseqs, except that transcripts with low LEIsc values (bottom 400) were chosen for set 1; each of the five substitution locations was analyzed separately (e.g., the matching took place only within a location). Once again, the mean PU of set 2 was set equal to unity for comparison. The actual PUs for ESSseqs in set 2 were 0.071 for WA, 0.126 for WD, 0.156 for HA, 0.120 for HM, and 0.053 for HD.

It was also explored whether the single strandedness of 3′SSs differed in substituted transcripts that had been induced to splice well compared with those with just average splicing. This analysis was restricted to locations WA and HA, which are close enough to the 3′SS to allow testing the effect of local folding. The PU of a 3′SS (the 15 nt from −14 to +1) was calculated as the average of the PUs of the 10 6-mers within it, and each calculated using the series of windows ranging from 28 to 66 nt; and the substituted 6-mer library position is required to be within the folding windows ranges considered. Two sets of transcripts were chosen for comparison: Set 1 was comprised of molecules with the top 400 LEIsc values (T400) and set 2 molecules were randomly drawn from transcripts with average LEIsc values (middle 1000). On a one-to-one basis, each 6-mer substitution chosen for set 2 had to match the G+C content of a ti-mer substitution in set 1. The mean PU of set 2 was set equal to unity for comparison. The same procedure was used for transcripts comprising the bottom 400 LEIsc values (B400). The actual PUs for the 3′SSs in set 2 were 0.283 for WA T400, 0.528 for HA T400, 0.244 for WA B400, and 0.579 for HA B400.

The single-strandedness of 5′SSs was measured analogously. This analysis was restricted to location WD, which is close enough to the 5′SS to allow testing the effect of local folding. The PU of a 5′SS (9 nt from −3 to +6) was calculated as the average of the PUs of the four 6-mers within it, and each calculated using the series of windows ranging from 28 to 66 nt; the substituted 6-mer library position is required to be within the folding windows ranges considered. Two sets of transcripts were chosen for comparison exactly as for the 3′SS. The PUs for the 5′ SSs in set 2 were set equal to unity for comparisons and were actually 0.179 for WD T400 and 0.169 for WD B400.

It was found that for four of the five locations ESEseqs have a higher probability of being unpaired (PU) when present in transcripts with enhanced splicing as opposed to those exhibiting average splicing, and which were matched for G+C content. ESSseqs also have a higher PU when present in transcripts with silenced splicing as opposed to average splicing. These results suggest that many of these splicing regulatory elements, both positive and negative, act through the binding of factors that require accessible single-stranded sequences.

It was then asked whether the single-stranded state of the splice sites (SSs) could be influenced by the substitution of a nearby 6-mer. At both locations, we found that 3′SSs have a higher PU in transcripts with enhanced splicing and a lower PU in transcripts with silenced splicing compared with transcripts with average splicing. This finding suggests that occlusion of the 3′SS in a doublestranded structure dampens its activity, most likely by preventing access to spliceosomal and related factors. For the 5′SS, only the WD location lies within the local folding range. Surprisingly, it was found that 5′SSs have a lower PU in transcripts with enhanced splicing than in transcripts with average splicing. This represents a surprising bias toward a double-stranded state.

Combinatorial requirements. Combinatorial effects among motifs could play a role in explaining the remaining 30% of the variance where Equation 3 does not hold. If a motif was positively or negatively synergistic with another within the 16-nt summed region, then the observed splicing would be significantly higher or lower than predicted, respectively. Such synergies could result from interactions among factors binding within this region or from competition for overlapping binding sites. Using this definition 232 motifs that could form positive synergies and 262 motifs that could form negative synergies were identified (P-value <0.05, t-test; FDRs of 17.7% and 15.6%, respectively). Similar results were obtained using a Kolmogorov-Smirnov (K-S) test. Many of these motifs resemble the binding sites of the known splicing factors ASF, 9G8, SRp30c, and hnRNPs A1/A2, K, M, L, and F/H. All of the splicing factors mentioned are abundantly expressed in the HEK293 cell line based on microarray data. Splicing factors binding within the 16-nt substitution region could also be interacting with factors that bind outside of the substituted region, either elsewhere in the exon or in the introns. Such synergistic effects could be effective at one location but not at another, and so result in a high variance, a misclassification as a neutral rather than an ESRseq, and a failure to be accurately predicted by Equation 3. Saturation mutagenesis experiments using a similar high-throughput sequencing approach should allow us to identify the partnering sequences in these putative synergic pairs, both beyond the 16-nt substitution region and within it.

Chromatin influence. Several recent studies have reported that exons are associated with greater nucleosome densities and distinctive histone modifications and that perturbation of histone modification can affect alternative splicing. It is possible that some of the 6-mers act as ESEs by promoting nucleosome assembly or positioning at the test exon and vice versa. The data from all five locations consistently showed a good correspondence between LEIsc values and predicted nucleosome occupancy scores as described by Kaplan N, Moore I K, Fondufe-Mittendorf Y, Gossett A J, Tillo D, Field Y, LeProustEM, Hughes T R, Lieb J D, Widom J, et al. “The DNA-encoded nucleosome organization of a eukaryotic genome,” Nature v458: pp 362-366 (2009), leaving open the possibility that chromatin structure is playing a role in the splicing enhancement seen here.

### 5. Analysis of Gene-Sequencing Data

Having identified 6-mer members that serve as splicing enhancers and inhibitors, it is possible to see their effects on other gene sequencing data to generalize the effect of the members on the splicing activity, e.g., in step 217 or 1313. ESEseqs as defined above exhibit a sharply higher abundance in exons compared with their intronic flanks, while ESS segs show the opposite behavior.

Previous gene-sequencing data is divided among different categories for these comparisons. Human mRNA sequences and ESTs were downloaded from the UniGene database and were aligned to the assembled genomic sequences (hg18) obtained from genomes/H_sapiens/using Sim4. Only ESTs that spanned at least two exon-exon junctions were used. Genes that exhibited no intron-exon junctions were excluded. Exons with no evidence of skipping or alternative splice site use were identified as constitutive exons. An exon that was excluded in one or more transcripts and present in at least one transcript was defined as an alternative cassette exon. Only exons flanked by canonical AG and GT dinucleotides were included. Pseudo exons were defined as intronic sequences having lengths between 50 and 250 nt and consensus values of ≧75 for 3′ splice sites and ≧78 for 5′ splice sites. The consensus values (CV) were based on a position-specific weight matrix and were calculated essentially according to Shapiro M B, Senaphthy P. “RNA splice junctions of different classes of eukaryotes: sequence statistics and functional implications in gene expression,” Nucleic Acids Res v15 pp 7155-7174 (1987). In addition, pseudo exons had to be at least 100 nt away from the closest real exon.

For genome-wide 6-mer density analysis, the exon lengths of human constitutive exons and alternative cassette exons were required to be at least 50 nt and the lengths of both flanking introns to be at least 100 nt. The total numbers of qualified constitutive exons and alternative cassette exons were 119,006 and 25,807, and the total number of pseudo exons (repeat-free) was 134,994. For a composite exon body, 50 nt were extracted from each end of each exon. For the two composite flanking introns, the 86-nt upstream and 94-nt downstream intronic sequences were extracted (excluding the 3′ and 5′ splice-site sequences). The 6-mers were enumerated starting at the borders of the splice-site sequences (−14 to +1 for the 3′SS and −3 to +6 for the 5′SS.

This enrichment/depletion is somewhat lower in alternative cassette exons compared with constitutive exons, and is not seen in pseudo exons. In addition, using the ratio of abundance in exons divided by abundance in intronic flanks as a sign of enhancer function, the top ESEseqs consistently outperformed the top 6-mers derived from LEIscs at individual locations; the same was true, in reverse, for ESSseqs. ESEseqs are conserved in evolution and exhibit a lower SNP density compared with scrambled controls; the reverse is true for ESSseqs. Also surveyed were ESRseq scores of 6-mers in and around more than 100,000 human exons at single-nucleotide resolution. Scores were strikingly higher in exons compared with adjacent intronic sequences; alternative cassette exons exhibited a somewhat lower difference from constitutive exons, while pseudo exons showed no such difference. The differences between the average ESRseq scores of constitutive, alternative, and pseudo exons were all highly significant (P<10⁻¹⁴⁰).

The ESRseq scores were used as a yardstick to interpret previously published determinations of splicing elements. ESEseqs coincided with many ESEs defined by computation, by five functional SELEX studies, and by SR protein-binding SELEX studies. Likewise, ESSseqs coincided with ESSs defined computationally, by functional selection (FAShex3s), and by hnRNP A1 binding SELEX. This coincidence is all the more remarkable given that many of these predictors do not agree with each other. No significant overlap was found for SRp40 nor for PTB. Interestingly, these proteins have been reported to act as both enhancers and silencers. All of the splicing factors mentioned are abundantly expressed in the HEK293 cell line based on microarray data.

While the overlap with all classes of previously described splicing regulatory sequences is highly significant, there are also a large number of ESRseqs that do not appear on previous lists. This result is not so surprising, since the SELEX-based methods yield only the best performers and the computationally derived sequences have been predicted with great conservatism (low P-value cutoffs) due to high noise and the desire to maximize validation.

A set of 58 human mutations known to affect splicing were also examined. 83% could be explained by a change in an ESRseq score in the predicted direction, compared with 33% for 39 mutations not affecting splicing and 51% for a random simulation of point mutations. Finally, ESRseq scores were applied to the extensive data of Goren A, Ram O, Amit M, Keren H, Lev-Maor G, Vig I, Pupko T, Ast G. “Comparative analysis identifies exonic splicing regulatory sequences—The complex definition of enhancers and silencers,” Mol Cell v22, pp 769-781 (2006), who proposed a positional effect to explain consistent differences in splicing caused by the substitution of 7-mers throughout an exon. It was found here that 78% (14/18) of these changes could be explained by changes in ESRseq scores of 6-mers created in sequences that overlapped the substitution.

### 6. Alternative Embodiments

In alternative embodiments, one or more library molecules or product molecules or output molecules include one or more of the sequences described next.

It is known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” is known in the art to refer to the region between the translation initiation codon and the translation termination codon. It is also known in the art that variants can be produced through the use of alternative signals to start or stop transcription and that pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

In the context of various embodiments, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides that can hydrogen bond with each other. Thus, “specifically hybridizable” and “complementary” are terms that are used to indicate a sufficient degree of complementarity or precise pairing such that stable and specific binding occurs between the nucleic acid and the DNA or RNA target.

Various conditions of stringency can be used for hybridization as is described below. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1 6.3.6, which is incorporated by reference. Aqueous and nonaqueous methods are described in that reference and either can be used. Specific hybridization conditions referred to herein are as follows: 1) low stringency hybridization conditions in 6.times.sodium chloride/sodium citrate (SSC) at about 45° C., followed by two washes in 0.2.times.SSC, 0.1% SDS at least at 50.degree C. (the temperature of the washes can be increased to 55° C. for low stringency conditions); 2) medium stringency hybridization conditions in 6.times.SSC at about 45° C., followed by one or more washes in 0.2.times.SSC, 0.1% SDS at 60° C.; 3) high stringency hybridization conditions in 6.times.SSC at about 45° C., followed by one or more washes in 0.2.times.SSC, 0.1% SDS at 65° C.; and preferably 4) very high stringency hybridization conditions are 0.5M sodium phosphate, 7% SDS at 65° C., followed by one or more washes at 0.2.times.SSC, 1% SDS at 65° C. Very high stringency conditions (4) are the preferred conditions and the ones that should be used unless otherwise specified.

Nucleic acids in the context of various embodiments include “oligonucleotides,” which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. DNA/RNA chimeras are also included.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure; however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Oligonucleotides containing modified backbones or non-natural internucleoside linkages can be used. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides. Preferred modified oligonucleotide backbones include, for example, phosphorothioates, chiral phosphorothioates, phosphorodithioates, phosphotriesters, aminoalkyl-phosphotriesters, methyl and other alkyl phosphonates including 3-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference. Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In some oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Some embodiments of some embodiments use oligonucleotides with phosphorothioate backbones and oligonucleosides with heteroatom backbones, and in particular —CH₂—NH—O—CH₂—, —CH₂—N(CH₃)—O—CH₂—[known as a methylene(methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂).sub.nNH₂, O(CH₂)ₙCH₃, O(CH₂)ₙONH₂, and O(CH₂)ₙON[(CH₂).sub.nCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy(2′—O—CH₂CH₂OCH₃, also known as 2′—O-(2-methoxyethyl) or 2′-MOE) (Martin et al., Hely. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethylamino-ethoxyethyl or 2′-DMAEOE), i.e., 2′—O—CH₂—O—CH₂—N(CH₂)₂, also described in examples hereinbelow.

A further modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Other modifications include 2′-methoxy(2′—O—CH₃), 2′-aminopropoxy (2′—OCH₂CH₂CH₂NH₂), 2′-allyl (2′—CH₂—CH═CH₂), 2′-O-allyl (2′—O—CH₂—CH═CH₂) and 2′-fluoro(2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine. (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C.ident.C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of some embodiments. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another modification of the oligonucleotides for use in some embodiments involves chemically linking to the oligonucleotide one or more moieties or conjugates which enhance the activity, cellular distribution or cellular uptake of the oligonucleotide. The compounds of some embodiments can include conjugate groups covalently bound to functional groups such as primary or secondary hydroxyl groups. Conjugate groups of some embodiments include intercalators, reporter molecules, polyamines, polyamides, poly ethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of various embodiments, include groups that improve oligomer uptake, enhance oligomer resistance to degradation, and/or strengthen sequence-specific hybridization with RNA. Groups that enhance the pharmacokinetic properties, in the context of various embodiments, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et. al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid; e.g., di hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of some embodiments may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. “Chimeric” compounds or “chimeras,” in the context of various embodiments, are oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids.

The oligonucleotides used in accordance with various embodiments may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

### 7. Computational Hardware Overview

FIG. 8 is a block diagram that illustrates a computer system 800 upon which an embodiment of the invention may be implemented. Computer system 800 includes a communication mechanism such as a bus 810 for passing information between other internal and external components of the computer system 800. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 800, or a portion thereof, constitutes a means for performing one or more steps of one or more methods described herein.

A sequence of binary digits constitutes digital data that is used to represent a number or code for a character. A bus 810 includes many parallel conductors of information so that information is transferred quickly among devices coupled to the bus 810. One or more processors 802 for processing information are coupled with the bus 810. A processor 802 performs a set of operations on information. The set of operations include bringing information in from the bus 810 and placing information on the bus 810. The set of operations also typically include comparing two or more units of information, shifting positions of units of information, and combining two or more units of information, such as by addition or multiplication. A sequence of operations to be executed by the processor 802 constitute computer instructions.

Computer system 800 also includes a memory 804 coupled to bus 810. The memory 804, such as a random access memory (RAM) or other dynamic storage device, stores information including computer instructions. Dynamic memory allows information stored therein to be changed by the computer system 800. RAM allows a unit of information stored at a location called a memory address to be stored and retrieved independently of information at neighboring addresses. The memory 804 is also used by the processor 802 to store temporary values during execution of computer instructions. The computer system 800 also includes a read only memory (ROM) 806 or other static storage device coupled to the bus 810 for storing static information, including instructions, that is not changed by the computer system 800. Also coupled to bus 810 is a non-volatile (persistent) storage device 808, such as a magnetic disk or optical disk, for storing information, including instructions, that persists even when the computer system 800 is turned off or otherwise loses power.

Information, including instructions, is provided to the bus 810 for use by the processor from an external input device 812, such as a keyboard containing alphanumeric keys operated by a human user, or a sensor. A sensor detects conditions in its vicinity and transforms those detections into signals compatible with the signals used to represent information in computer system 800. Other external devices coupled to bus 810, used primarily for interacting with humans, include a display device 814, such as a cathode ray tube (CRT) or a liquid crystal display (LCD), for presenting images, and a pointing device 816, such as a mouse or a trackball or cursor direction keys, for controlling a position of a small cursor image presented on the display 814 and issuing commands associated with graphical elements presented on the display 814.

In the illustrated embodiment, special purpose hardware, such as an application specific integrated circuit (IC) 820, is coupled to bus 810. The special purpose hardware is configured to perform operations not performed by processor 802 quickly enough for special purposes. Examples of application specific ICs include graphics accelerator cards for generating images for display 814, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 800 also includes one or more instances of a communications interface 870 coupled to bus 810. Communication interface 870 provides a two-way communication coupling to a variety of external devices that operate with their own processors, such as printers, scanners and external disks. In general the coupling is with a network link 878 that is connected to a local network 880 to which a variety of external devices with their own processors are connected. For example, communication interface 870 may be a parallel port or a serial port or a universal serial bus (USB) port on a personal computer. In some embodiments, communications interface 870 is an integrated services digital network (ISDN) card or a digital subscriber line (DSL) card or a telephone modem that provides an information communication connection to a corresponding type of telephone line. In some embodiments, a communication interface 870 is a cable modem that converts signals on bus 810 into signals for a communication connection over a coaxial cable or into optical signals for a communication connection over a fiber optic cable. As another example, communications interface 870 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 870 sends and receives electrical, acoustic or electromagnetic signals, including infrared and optical signals, that carry information streams, such as digital data.

The term computer-readable medium is used herein to refer to any medium that participates in providing information to processor 802, including instructions for execution. Such a medium may take many forms, including, but not limited to, non-volatile media, volatile media and transmission media. Non-volatile media include, for example, optical or magnetic disks, such as storage device 808. Volatile media include, for example, dynamic memory 804. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 802, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read.

Logic encoded in one or more tangible media includes one or both of processor instructions on a computer-readable storage media and special purpose hardware, such as ASIC *820.

Network link 878 typically provides information communication through one or more networks to other devices that use or process the information. For example, network link 878 may provide a connection through local network 880 to a host computer 882 or to equipment 884 operated by an Internet Service Provider (ISP). ISP equipment 884 in turn provides data communication services through the public, world-wide packet-switching communication network of networks now commonly referred to as the Internet 890. A computer called a server 892 connected to the Internet provides a service in response to information received over the Internet. For example, server 892 provides information representing video data for presentation at display 814.

The invention is related to the use of computer system 800 for implementing the techniques described herein. According to one embodiment of the invention, those techniques are performed by computer system 800 in response to processor 802 executing one or more sequences of one or more instructions contained in memory 804. Such instructions, also called software and program code, may be read into memory 804 from another computer-readable medium such as storage device 808. Execution of the sequences of instructions contained in memory 804 causes processor 802 to perform the method steps described herein. In alternative embodiments, hardware, such as application specific integrated circuit 820, may be used in place of or in combination with software to implement the invention. Thus, embodiments of the invention are not limited to any specific combination of hardware and software.

The signals transmitted over network link 878 and other networks through communications interface 870, carry information to and from computer system 800. Computer system 800 can send and receive information, including program code, through the networks 880, 890 among others, through network link 878 and communications interface 870. In an example using the Internet 890, a server 892 transmits program code for a particular application, requested by a message sent from computer 800, through Internet 890, ISP equipment 884, local network 880 and communications interface 870. The received code may be executed by processor 802 as it is received, or may be stored in storage device 808 or other non-volatile storage for later execution, or both. In this manner, computer system 800 may obtain application program code in the form of a signal on a carrier wave.

Various forms of computer readable media may be involved in carrying one or more sequence of instructions or data or both to processor 802 for execution. For example, instructions and data may initially be carried on a magnetic disk of a remote computer such as host 882. The remote computer loads the instructions and data into its dynamic memory and sends the instructions and data over a telephone line using a modem. A modem local to the computer system 800 receives the instructions and data on a telephone line and uses an infra-red transmitter to convert the instructions and data to a signal on an infra-red a carrier wave serving as the network link 878. An infrared detector serving as communications interface 870 receives the instructions and data carried in the infrared signal and places information representing the instructions and data onto bus 810. Bus 810 carries the information to memory 804 from which processor 802 retrieves and executes the instructions using some of the data sent with the instructions. The instructions and data received in memory 804 may optionally be stored on storage device 808, either before or after execution by the processor 802.

FIG. 9 illustrates a chip set 900 upon which an embodiment of the invention may be implemented. Chip set 900 is programmed to perform one or more steps of a method described herein and includes, for instance, the processor and memory components described with respect to FIG. 8 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 900, or a portion thereof, constitutes a means for performing one or more steps of a method described herein.

In one embodiment, the chip set 900 includes a communication mechanism such as a bus 901 for passing information among the components of the chip set 900. A processor 903 has connectivity to the bus 901 to execute instructions and process information stored in, for example, a memory 905. The processor 903 may include one or more processing cores with each core configured to perform independently. A multi-core processor enables multiprocessing within a single physical package. Examples of a multi-core processor include two, four, eight, or greater numbers of processing cores. Alternatively or in addition, the processor 903 may include one or more microprocessors configured in tandem via the bus 901 to enable independent execution of instructions, pipelining, and multithreading. The processor 903 may also be accompanied with one or more specialized components to perform certain processing functions and tasks such as one or more digital signal processors (DSP) 907, or one or more application-specific integrated circuits (ASIC) 909. A DSP 907 typically is configured to process real-world signals (e.g., sound) in real time independently of the processor 903. Similarly, an ASIC 909 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 903 and accompanying components have connectivity to the memory 905 via the bus 901. The memory 905 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 905 also stores the data associated with or generated by the execution of one or more steps of the methods described herein.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

1. A method comprising:
preparing a library of molecules that can be sequenced, wherein the library includes one or more instances of each possible member of a k-mer;
sequencing a first population of the library to determine the relative frequency of each member of the k-mer in a population of library molecules;
contacting a second population of the library with a biochemical system;
sequencing a population of output molecules to determine the relative frequency of each member of the k-mer in the population of output molecules, wherein each output molecule is related to a product of a process of the biochemical system and carries a k-mer related to a corresponding k-mer of a library molecule involved in the process; and
determining effectiveness of each member of the k-mer based on the relative frequency of each member of the k-mer in the population of output molecules and the relative frequency of the corresponding k-mer in the library.

2. A method as recited in claim 1, wherein determining the effectiveness further comprises determining a ratio of the relative frequency of each member of the k-mer in the population of output molecules to relative frequency in the library population of the corresponding k-mer.

3. A method as recited in claim 1, wherein the corresponding k-mer in the library is the same as the k-mer in the output molecule.

4. A method as recited in claim 1, wherein the k-mer is a sequence of k nucleotides and the corresponding k-mer in the library is complementary to the k-mer in the output molecule.

5. A method as recited in claim 1, wherein k-mer is a sequence of k amino acids.

6. A method as recited in claim 1, wherein the output molecule is the same as the product of the process of the biochemical system.

7. A method as recited in claim 1, wherein the output molecule is complementary DNA reverse transcribed from the product of the process of the biochemical system.

8. A method as recited in claim 1, wherein the output molecule is a phage display of a peptide product of the process of the biochemical system.

9. A method as recited in claim 1, wherein the output molecule is a ribosome display of a peptide product of the process of the biochemical system.

10. A method as in claim 1, wherein.
the library of molecule are pre-messenger ribonucleic acid (pre-mRNA) molecules;
contacting the library with a biochemical system comprises transfecting the library into living cells; and
the output molecules are mRNA molecules spliced by the cells from the pre-mRNA molecules.

11. A method as in claim 1, wherein.
the library of molecules comprises DNA molecules that code for a particular gene or fragment thereof;
contacting the library with a biochemical system comprises transfecting the library into living cells; and
the output molecules are complementary deoxyribonucleic acid (cDNA) of ribonucleic acid (RNA) spliced by the cells from pre-mRNA molecules derived from the DNA molecules.

12. A method as recited in claim 1, further comprising selecting a particular set of one or more members of the k-mer based on an effectiveness determined for the particular set.

13. A method as recited in claim 12, further comprising contacting a biochemical system with at least one member of the particular set of one or more members of the k-mer, to affect an outcome of a process of the biochemical system.

14. A method as recited in claim 1, wherein preparing the library further comprises synthesizing the library using polymerase chain reaction (PCR).

15. A method as recited in claim 1, wherein preparing the library further comprises synthesizing the library without using plasmids cloned in Escherichia coli cells.

16. A method as recited in claim 1, wherein.
the k-mer comprises 6 nucleotides;
the library comprises DNA; and
contacting the library with a biochemical system further comprises transfecting the library into more than about one million cells.

17. A method as recited in claim 14, wherein preparing the library further comprises using a human cytomegalovirus (CMV) promoter.

18. A method as recited in claim 14, wherein the first population of the library is the same as the second population of the library.

19. A method as recited in claim 2, wherein determining the effectiveness further comprises determining a distribution of the ratio over multiple locations of the k-mer in one or more input molecules.

20. A method as recited in claim 19, wherein determining the effectiveness further comprises determining an enhancing k-mer or inhibiting k-mer based on significantly different distributions of the ratio when the k-mer is present than when the k-mer is absent.

21. A method as recited in claim 20, wherein determining the effectiveness further comprises determining a net effect of a substitution of a k-mer at a particular location based on a relative occurrence of every enhancing k-mer and every inhibiting k-mer in a vicinity of the substitution.

22. A computer-readable storage medium carrying one or more sequences of instructions, wherein execution of the one or more sequences of instructions by one or more processors causes an apparatus to:
determine a relative frequency of each member of a k-mer in a population of library molecules;
determine the relative frequency of each member of the k-mer in a population of output molecules, wherein each output molecule is related to a product of a process of a biochemical system and carries a k-mer related to a corresponding k-mer of a library molecule involved in the process; and
determine effectiveness of each member of the k-mer based on the relative frequency of each member of the k-mer in the population of output molecules and the relative frequency of the corresponding k-mer in the library.

23. An apparatus comprising:
means for determining a relative frequency of each member of a k-mer in a population of library molecules;
means for determining the relative frequency of each member of the k-mer in a population of output molecules, wherein each output molecule is related to a product of a process of a biochemical system and carries a k-mer related to a corresponding k-mer of a library molecule involved in the process; and
means for determining effectiveness of each member of the k-mer based on the relative frequency of each member of the k-mer in the population of output molecules and the relative frequency of the corresponding k-mer in the library.


SEQUENCE LISTING (31 sequences)

SEQ ID NO: 1 - DNA, 21 bp, Artificial Sequence
  Synthetic oligonucleotide
agagtctgagatggcctggct

SEQ ID NO: 2 - DNA, 20 bp, Artificial Sequence
  Synthetic oligonucleotide
gtcagatccgcctccgcgta

SEQ ID NO: 3 - DNA, 20 bp, Artificial Sequence
  Synthetic oligonucleotide
gtaaacggaactgcctccaa

SEQ ID NO: 4 - DNA, 20 bp, Artificial Sequence
  Synthetic oligonucleotide
tgccacctgacgtctaagaa

SEQ ID NO: 5 - DNA, 60 bp, Artificial Sequence
  Synthetic oligonucleotide
ccatttcactgtgctggagctcccnnnnnnaactctagaaaagaagaagaggtggggagt

SEQ ID NO: 6 - DNA, 20 bp, Artificial Sequence
  Synthetic oligonucleotide
gctccagcacagtgaaatgg

SEQ ID NO: 7 - DNA, 20 bp, Artificial Sequence
  Synthetic oligonucleotide
ctcctgaaaatctcgccaag

SEQ ID NO: 8 - DNA, 54 bp, Artificial Sequence
  Synthetic oligonucleotide
caagcagaagacggcatacgagctcttccgatcttctagctgggagcaaagtcc

SEQ ID NO: 9 - DNA, 78 bp, Artificial Sequence
  Synthetic oligonucleotide
aatgatacggcgaccaccgagatctacactctttccctacacgacgctcttccgatctnnttcactgagctggagctc

SEQ ID NO: 10 - DNA, 54 bp, Artificial Sequence
  Synthetic oligonucleotide
caagcagaagacggcatacgagctcttccgatctaccgatccagcctccgcgta

SEQ ID NO: 11 - DNA, 6 bp, Artificial Sequence
  Synthetic oligonucleotide
gacgtc

SEQ ID NO: 12 - DNA, 6 bp, Artificial Sequence
  Synthetic oligonucleotide
ccagca

SEQ ID NO: 13 - DNA, 6 bp, Artificial Sequence
  Synthetic oligonucleotide
aaagag

SEQ ID NO: 14 - DNA, 71 bp, Artificial Sequence
  Synthetic oligonucleotide
ttcttcttttctagagttnnnnnngggagctccagcacagtgaaatggacagaagggcagagcaagtgagt

SEQ ID NO: 15 - DNA, 16 bp, Artificial Sequence
  Synthetic oligonucleotide
gagttgacgtcgggag

SEQ ID NO: 16 - DNA, 16 bp, Artificial Sequence
  Synthetic oligonucleotide
aatggacgtcagggca

SEQ ID NO: 17 - DNA, 16 bp, Artificial Sequence
  Synthetic oligonucleotide
aatggacgtcagggca

SEQ ID NO: 18 - DNA, 16 bp, Artificial Sequence
  Synthetic oligonucleotide
aatggacgtcggggca

SEQ ID NO: 19 - DNA, 16 bp, Artificial Sequence
  Synthetic oligonucleotide
aatggacgtctgggca

SEQ ID NO: 20 - DNA, 16 bp, Artificial Sequence
  Synthetic oligonucleotide
aatgggacgtcgggca

SEQ ID NO: 21 - DNA, 16 bp, Artificial Sequence
  Synthetic oligonucleotide
tacccagacgtccaga

SEQ ID NO: 22 - DNA, 16 bp, Artificial Sequence
  Synthetic oligonucleotide
taccccgacgtccaga

SEQ ID NO: 23 - DNA, 16 bp, Artificial Sequence
  Synthetic oligonucleotide
tacccgacgtcccaga

SEQ ID NO: 24 - DNA, 16 bp, Artificial Sequence
  Synthetic oligonucleotide
tacccggacgtccaga

SEQ ID NO: 25 - DNA, 16 bp, Artificial Sequence
  Synthetic oligonucleotide
taccctgacgtccaga

SEQ ID NO: 26 - DNA, 16 bp, Artificial Sequence
  Synthetic oligonucleotide
tggcagacgtctgctc

SEQ ID NO: 27 - DNA, 16 bp, Artificial Sequence
  Synthetic oligonucleotide
ctgtgacgtcatgcac

SEQ ID NO: 28 - DNA, 16 bp, Artificial Sequence
  Synthetic oligonucleotide
ctgtgacgtcctgcac

SEQ ID NO: 29 - DNA, 16 bp, Artificial Sequence
  Synthetic oligonucleotide
ctgtgacgtcgtgcac

SEQ ID NO: 30 - DNA, 16 bp, Artificial Sequence
  Synthetic oligonucleotide
ctgtgacgtcttgcac

SEQ ID NO: 31 - DNA, 16 bp, Artificial Sequence
  Synthetic oligonucleotide
ctgtggacgtctgcac